(19) United States
(12) Patent Application Publication
(10) Pub. No.: US 2022/0330946 A1
(43) Pub. Date: Oct. 20, 2022

(54) DEVICES AND METHODS FOR AT LEAST PARTIALLY OCCLUDING A BLOOD VESSEL WHILE MAINTAINING DISTAL PERFUSION

(71) Applicant: RENALPRO MEDICAL, INC., Santa Clara, CA (US)

(72) Inventors: Nicholas DeBEER, Montara, CA (US); Daniel LEONG LEONG, San Mateo, CA (US)

(73) Assignee: RENALPRO MEDICAL, INC., Santa Clara, CA (US)

(21) Appl. No.: 17/641,854

(22) PCT Filed: Sep. 25, 2020
(86) PCT No.: PCT/US2020/052899

§ 371 Date: Mar. 10, 2022

Related U.S. Application Data
(60) Provisional application No. 62/905,874, filed on Sep. 25, 2019.

(51) Int. Cl. A61B 17/12; A61M 60/139

(52) U.S. Cl. CPC A61B 17/12109; A61B 17/12031; A61B 17/12036; A61B 17/1204; A61B 17/12136; A61B 17/12168; A61M 60/139

(57) ABSTRACT

Temporary vascular occlusion devices and methods for use thereof are described which provide temporary vascular occlusion while maintaining distal perfusion. The temporary vascular occlusion device may include a multiple layer scaffold covering having proximal and distal attachment zones separated by an unattached scaffold covering zone where the scaffold covering is adjacent to but not attached directly to the scaffold frame.

### CROSS REFERENCE TO RELATED APPLICATIONS

This application claims priority to U.S. Provisional Patent Application No. 62/905,874, filed Sep. 25, 2019, titled “DEVICES AND METHODS FOR AT LEAST PARTIALLY OCCLUDING A BLOOD VESSEL WHILE MAINTAINING DISTAL PERFUSION,” which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

This application relates to various methods and devices for at least partially occluding peripheral blood flow from a blood vessel while maintaining perfusion to blood vessels and structures distal to the occlusion site.

### BACKGROUND

Acute kidney injury (AKI), also called acute renal failure (ARF), is a rapid loss of kidney function. Its causes are numerous and include low blood volume from any cause, exposure to substances harmful to the kidney, and obstruction of the urinary tract. AKI is diagnosed on the basis of characteristic laboratory findings, such as elevated blood creatinine, or inability of the kidneys to produce sufficient amounts of urine.

Acute kidney injury is diagnosed on the basis of clinical history and laboratory data. A diagnosis is made when there is rapid reduction in kidney function, as measured by serum creatinine, or based on a rapid reduction in urine output, termed oliguria.

For example, the use of intravascular iodinated contrast agents may cause acute kidney injury. In patients receiving intravascular iodine-containing contrast media for angiography, contrast-induced AKI (CI-AKI) is a common problem and is associated with excessive hospitalization cost, morbidity, and mortality. Clinical procedures involving intravascular iodine-containing contrast media injection include for example, percutaneous coronary intervention (PCI), peripheral vascular angiography and intervention, neurological angiography and intervention. Solutions have been suggested for occluding at least partially the blood flow into the renal arteries during procedures where a patient is exposed to intravascular contrast.

While some solutions have been proposed for vascular occlusion, the need for improved methods and devices remain.

### SUMMARY OF THE DISCLOSURE

In one aspect provides a device for treating or reduce the risk of acute kidney injury or to provide temporary partial or total occlusion of a blood vessel, comprising: an at least partially covered scaffold on a distal portion of a catheter. The covering or membrane or coating on the scaffold structure provides a functional aspect similar to the disturbing means examples described herein which are associated with a balloon embodiment. In use, the at least partially covered scaffold structure may be positioned to allow some flow, occlude all flow or modulate between flow, no flow or partial flow conditions based on the position of the scaffold structure relative to the blood vessel interior wall.

In another aspect provides a temporary occlusion device for at least partially occluding some or all peripheral vessels from a blood vessel while allowing perfusion to distal vessels and structures. In use when the blood vessel is an aorta, the temporary occlusion device is a partially covered scaffold with an optional position indicator wherein the partially covered scaffold is deployed to occlude completely or partially one or more of a blood vessel in the aorta, the suprarenal aorta or the infrarenal aorta. In another aspect, the at least partially covered scaffold structure is deployed within an aorta to occlude partially or completely one or more or a combination of: a hepatic artery, a gastric artery, a celiac trunk, a splenic artery, an adrenal artery, a renal artery, a superior mesenteric artery, an ileocolic artery, a gonadal artery and an inferior mesenteric artery while simultaneously allowing perfusion flow through or around the at least partially covered scaffold structure to distal vessels and structures.

In some embodiments, the insertion of the at least partially covered scaffold device to an aorta is applied either by transfemoral artery approach or by trans-brachial artery approach or by trans-radial artery approach. In certain embodiments, the catheter further includes an inner shaft adapted for use with a guidewire. In certain embodiments, the method further comprises initially inserting a guidewire into a vessel leading to an aorta.

In general, in one embodiment, a vascular occlusion device includes a handle having a slider, an inner shaft coupled to the handle, an outer shaft over the inner shaft and coupled to the slider, a scaffold structure having a distal end, a scaffold transition zone and a proximal end having a plurality of legs wherein each leg of the plurality legs is coupled to a distal portion of the inner shaft. The scaffold structure moves from a stowed configuration when the outer shaft is extended over the scaffold structure and a deployed configuration when the outer shaft is retracted from covering the scaffold structure. There may be a multiple layer scaffold covering over at least a portion of the scaffold structure. The multiple layer scaffold covering has a distal scaffold attachment zone where a portion of the scaffold covering is attached to a distal portion of the scaffold, a proximal scaffold attachment zone where a portion of the scaffold covering is attached to a proximal portion of the scaffold. There is also an unattached zone between the distal attachment zone and the proximal attachment zone where the scaffold covering is unattached to an adjacent portion of the scaffold.

This and other embodiments include one or more of the following features. The plurality of legs can be two legs or three legs. The scaffold covering can extend from the distal end of the scaffold structure to each of the two legs or the three legs. The scaffold covering can extend from the distal end of the scaffold structure proximally to cover approximately 20%, 50%, 80% or 100% of the overall length of the scaffold structure. The scaffold covering can extend completely circumferentially about the scaffold structure from the distal attachment zone to the proximal attachment zone. The scaffold covering can extend partially circumferentially about the scaffold structure from the distal attachment zone to the proximal attachment zone with an uncovered scaffold structure. The scaffold covering can extend partially circumferentially about 270 degrees of the scaffold structure from the distal attachment zone to the proximal attachment zone. A first scaffold covering can extend partially circumferentially about 45 degrees of the scaffold structure from the distal attachment zone to the proximal attachment zone and a second scaffold covering can extend partially circumferentially about 45 degrees of the scaffold structure from the distal attachment zone to the proximal attachment zone. The first scaffold covering and the second scaffold covering can be on opposite sides of the longitudinal axis of the scaffold structure. The multiple layer scaffold covering can be attached to the scaffold in the distal scaffold attachment zone and in the proximal scaffold attachment zone by encapsulating a portion of the scaffold, by folding over a portion of the multiple layer scaffold covering and encapsulating a portion of the scaffold, by stitching the multiple layer scaffold covering to a portion of the scaffold, or by electrospinning the multiple layer scaffold to a portion of the scaffold. The scaffold structure can be formed from slots cut into a tube. The covering can be applied to nearly all, 80%, 70%, 60%, 50%, 30% or 20% of the scaffold structure. Scaffold covering can be formed from multiple layers. The layers of the multiple layer scaffold covering can be selected from ePFTE, PTFE, FEP, polyurethane or silicone. The scaffold covering or the more than one layers of a multiple layer scaffold covering can be applied to a scaffold structure external surface, to a scaffold structure internal surface, to encapsulate the distal scaffold attachment zone and the proximal scaffold attachment zone, as a series of spray coats, dip coats or electron spin coatings to the scaffold structure. The multiple layer scaffold covering can have a thickness of 5-100 microns. The multiple layer scaffold covering can have a thickness of about 0.001 inches in an unattached zone and a thickness of about 0.002 inches in an attached zone. The vascular occlusion can further include a double gear pinion within the handle that couples the outer shaft to the slider.

In general, in one embodiment, a method of providing selective occlusion with distal perfusion using a vascular occlusion device includes: (1) advancing the vascular occlusion device in a stowed condition along a blood vessel to a position adjacent to one or more peripheral blood vessels in the portion of the vasculature of the patient selected for occlusion while the vascular occlusion device is tethered to a handle outside of the patient; (2) transitioning the vascular occlusion device from the stowed condition to a deployed condition using the handle wherein the vascular occlusion device at least partially occludes blood flow into the one or more peripheral blood vessels selected for occlusion wherein the position of the vascular occlusion device engages with a superior aspect of the vasculature to direct blood flow into and along a lumen defined by a covered scaffold structure of the vascular occlusion device; (3) deflecting a portion of an unattached zone of the covered scaffold in response to the blood flow through the lumen of the covered scaffold into an adjacent opening of the one or more peripheral blood vessels in the portion of the vasculature of the patient selected for occlusion; (4) transitioning the vascular occlusion device from the deployed condition to the stowed condition using the handle; and (5) withdrawing the vascular occlusion device in the stowed condition from the patient.

This and other embodiments can include one or more of the following features. The one or more peripheral blood vessels in the portion of the vasculature of the patient selected for occlusion can be selected from the group consisting of a hepatic artery, a gastric artery, a celiac trunk, a splenic artery, an adrenal artery, a renal artery, a superior mesenteric artery, an ileocolic artery, a gonadal artery and an inferior mesenteric artery. The covered scaffold unattached zone can further include a position of a portion of the unattached zone to deflect into a portion of at least one of a hepatic artery, a gastric artery, a celiac trunk, a splenic artery, an adrenal artery, a renal artery, a superior mesenteric artery, an ileocolic artery, a gonadal artery and an inferior mesenteric artery when the vascular occlusion device is positioned within a portion of the aorta.

In general, in one embodiment, a method of temporarily occluding a blood vessel includes: (1) advancing a vascular occlusion device in a stowed condition along a blood vessel to a position adjacent to one or more peripheral blood vessels selected for temporary occlusion; (2) transitioning the vascular occlusion device from the stowed condition to a deployed condition wherein the vascular occlusion at least partially occludes blood flow into the one or more peripheral blood vessels selected for temporary occlusion while directing the blood flow through and along a lumen of a covered scaffold of the vascular occlusion device; and (3) transitioning the vascular occlusion device out of the deployed condition to restore blood flow into the one or more peripheral blood vessels selected for temporary occlusion when a period of temporary occlusion is elapsed.

This and other embodiments can include one or more of the following features. Directing the blood flow through and along the lumen of the vascular occlusion device can maintain blood flow to components and vessels distal to the vascular occlusion device while at least partially occluding the blood flow to the one or more peripheral blood vessels. The one or more peripheral blood vessels can be the vasculature of a liver, a kidney, a stomach, a spleen, an intestine, a stomach, an esophagus, or a gonad. The blood vessel can be an aorta and the peripheral blood vessels are one or more or a combination of: a hepatic artery, a gastric artery, a celiac trunk, a splenic artery, an adrenal artery, a renal artery, a superior mesenteric artery, an ileocolic artery, a gonadal artery and an inferior mesenteric artery.

In general, in one embodiment, a method of reversibly and temporarily occluding a blood vessel includes: (1) advancing an at least partially covered scaffold structure of a tethered vascular occlusion device to a portion of an aorta to be occluded; and (2) using a handle of the vascular occlusion device to deploy the at least partially covered scaffold structure within the aorta to occlude partially or completely one or more or a combination of: a hepatic artery, a gastric artery, a celiac trunk, a splenic artery, an adrenal artery, a renal artery, a superior mesenteric artery, an ileocolic artery, a gonadal artery and an inferior mesenteric artery using a portion of a multiple layer scaffold covering while simultaneously allowing perfusion flow through a lumen of the at least partially covered scaffold structure to distal vessels and structures.

This and other embodiments can include one or more of the following features. The insertion of the vascular occlusion device or of the at least partially covered scaffold device to a blood vessel which is the aorta can be introduced by transfemoral artery approach or by trans-brachial artery approach or by trans-radial artery approach. The method can further include advancing the vascular occlusion device over a guidewire into a position adjacent to a landmark of the skeletal anatomy. A portion of an unattached zone of a multiple layer scaffold covering can distend in response to blood flow along a lumen of the scaffold of the vascular occlusion device to occlude an opening of any of a hepatic artery, a gastric artery, a celiac trunk, a splenic artery, an adrenal artery, a renal artery, a superior mesenteric artery, an ileocolic artery, a gonadal artery and an inferior mesenteric artery.

In general, in one embodiment, a vascular occlusion device include a handle having a slider knob, an inner shaft coupled to the handle, an outer shaft over the inner shaft and coupled within the handle to the slider knob, a scaffold structure having at least two legs and a multiple layer scaffold covering, and the multiple layer scaffold covering positioned over at least a portion of the scaffold structure. The at least two legs of the scaffold structure are attached to an inner shaft coupler in a distal portion of the inner shaft. The scaffold structure moves from a stowed condition when the outer shaft is extended over the scaffold structure and a deployed condition when the outer shaft is retracted from covering the scaffold structure.

This and other embodiments can include one or more of the following features. The scaffold structure can be formed from slots cut into a tube. The covering can be applied to nearly all, 80%, 70%, 60%, 50%, 30% or 20% of the scaffold structure. The multiple layer scaffold covering can be made of ePFTE, PTFE, polyurethane, FEP or silicone. The multiple layer scaffold covering can be folded over a proximal portion and a distal portion of the scaffold. After the multiple layer scaffold covering is attached to the scaffold, the scaffold can further include a distal attachment zone, a proximal attachment zone and an unattached zone. The multiple layer scaffold covering can further include a proximal attachment zone, a distal attachment zone and an unattached zone wherein a thickness of the multiple layer covering in the proximal attachment zone and the distal attachment zone is greater than the thickness of the multiple layer scaffold covering in the unattached zone. The multiple layer scaffold covering on the scaffold structure can have a thickness of 5-100 microns. Scaffold structure can have a cylindrical portion and a conical portion. The terminal ends of the conical portion can be coupled to the inner shaft. The inner shaft can further include one or more spiral cut sections to increase flexibility of the inner shaft. The one or more spiral cut sections can be positioned proximally or distally or both proximal and distal to an inner shaft coupler where the scaffold structure is attached to the inner shaft. The scaffold structure can further include two or more legs. Each of the two or more legs can terminate with a connection tab that is joined to a corresponding key feature on an inner shaft coupler. The multiple layer scaffold covering can include one or more or a pattern of apertures that are shaped, sized or positioned relative to the scaffold structure to modify the amount of distal perfusion provided by the vascular occlusion device in use within the vasculature. The multiple layer scaffold covering can include one or more regular or irregular geometric shapes arranged in a continuous or discontinuous pattern which is selected to adapt the distal perfusion flow profile of the vascular occlusion device in use within the vasculature. When in a stowed configuration within the outer shaft the overall diameter can be between 0.100 inches and 0.104 inches and when in a deployed configuration the covered scaffold has an outer diameter from 19 to 35 mm. The covered scaffold can have an occlusive length of 40 mm to 100 mm measured from a distal end of the scaffold to a scaffold transition zone.

### BRIEF DESCRIPTION OF THE DRAWINGS

A better understanding of the features and advantages of the present invention will be obtained by reference to the following detailed description that sets forth illustrative embodiments, in which the principles of the invention are used, and the accompanying drawings of which:

FIG. 1 illustrates a diagram of an exemplary invention device comprises a balloon catheter having a first balloon positioned at the supra-renal aorta position near orifices of bilateral renal arteries for treating acute kidney injury.

FIG. 2 illustrates a diagram of an exemplary invention device for treating acute kidney injury where first balloon is inflated to occlude the orifice of both sides of renal arteries.

FIGS. 3A to 3D are perspective views of first balloon of the invention device. FIG. 3A shows an cylinder-like inflated balloon. FIG. 3C shows the morphology of an exemplary inflated first balloon which is “butter-fly like.” FIG. 3B shows a cross-section view of the cylinder-like inflated balloon of FIG. 3A. FIG. 3D shows a cross-section view of the cylinder-like inflated balloon of FIG. 3B.

FIG. 4 illustrates a diagram showing deflated first balloon 402 and a second balloon 403 is inflated at the location of infra-renal aorta near the orafice of renal arteries.

FIG. 5 illustrates a diagram showing the vortex blood flow caused by 2^{nd} balloon distension.

FIG. 6 shows that a normal saline can be infused from a control box, through the catheter pore 606 into the supra-renal aorta while a second balloon remain inflated.

FIG. 7 shows another aspect of the invention where the first balloon exerts renal artery blood flow augmentation by periodic inflation and deflation of the first balloon.

FIG. 8 shows at the end of PCI, both the first and second balloons are deflated and normal saline as postprocedural hydration continuous infusion.

FIG. 9 shows another aspect of the present invention, where a guidewire is used to guide the device for insertion into the renal artery.

FIG. 10 shows that a spinning propeller is inserted to renal artery and then spins around the central guide wire to augment renal artery blood flow toward the kidney.

FIGS. 11A-11B show variation embodiments of a spinning propeller.

FIGS. 12A-12C shows another embodiment of invention disturbing means where a cone shaped wire device 1702 partially covered with tunnel membrane 1703 which is deployed from catheter 1701. FIG. 12A shows a side cross section view of an exemplary wire device 1702.

FIG. 12B shows the specification of the exemplary wire device 1702 in aorta. FIG. 12C shows that a normal saline or other suitable medicine can be applied via an injection hole (or holes) 1708 via an infusion tube 1707 at the distal opening 1704 or the proximal opening 1705, or combination thereof.

FIGS. 13A-13D illustrate a variation of the embodiment of FIGS. 12A-12C where a cone-cylinder shaped wire device 1802 partially covered with tunnel membrane 1803 is shown. FIG. 13A show a side cross section view of the wire device 1802. FIG. 13B shows a top view of the wire device 1802. FIG. 13C shows a bottom view of the wire device 1802. FIG. 13D provides an isometric view of the wire device 1802.

FIGS. 14A-14C show yet another embodiment of the present disclosure. FIG. 14A shows a catheter shaft comprising an outer shaft, an inner shaft disposed therein. FIG. 14B shows the catheter shaft device with expandable mesh braid coupled to the inner and outer shafts in a low-profile configuration. FIG. 14C shows the catheter shaft device with expandable mesh braid in an expanded configuration.

FIGS. 14D-14G show further embodiments of the present disclosure. FIG. 14D shows a prototype of a catheter shaft device with expandable mesh braid. FIG. 14E shows a fully open mesh braid. FIG. 14F shows a partially collapsed mesh braid. FIG. 14G shows a fully collapsed mesh braid.

FIGS. 15A-15D show the deployment of the embodiment of FIGS. 14A-14G. FIG. 15A shows the insertion of the embodiment into the abdominal aorta. FIG. 15B shows the positioning of the device in the abdominal aorta. FIG. 15C shows the device deployed. FIG. 15D shows the device collapsed.

FIG. 16 is a distal end view of a bare scaffold showing three legs each terminating in a connection tab.

FIG. 17 is an isometric view of the bare scaffold of FIG. 16.

FIG. 18 is a side view of an exemplary scaffold structure having two legs only one visible in this view.

FIG. 19 is a side view of a bare scaffold with two legs for attachment to an inner shaft.

FIG. 20 is an enlarged view of the connection tab on the end of each of the two legs of the scaffold embodiment of FIG. 19.

FIGS. 21A and 21B are side and perspective views, respectively, of the two key features of an inner shaft coupler that is attached to an inner shaft.

FIG. 21C is an enlarged view of the shaft coupler of FIGS. 21A and 21B showing the detail of a key feature shaped to engage with a connection tab of a scaffold leg.

FIG. 22 is a side view of the two connection tabs of the scaffold legs of the scaffold of FIGS. 19 and 20 engaged with the inner shaft coupler of FIGS. 21A-21C.

FIG. 23A is an exemplary scaffold attached to an inner shaft coupler of an inner shaft having a plurality of spiral cuts.

FIG. 23B is an enlarged view of the scaffold in FIG. 23A showing the spiral cut detail in the distal portion of the inner shaft.

FIG. 24A is an exemplary view of a covered scaffold in a deployed configuration connected to the inner shaft. Openings cut around the legs and the atraumatic tip of the inner shaft are also visible in this view.

FIG. 24B is an enlarged view of the proximal end of the covered scaffold in FIG. 24A showing the covering on the legs extends into the inner shaft coupler. This view also shows the cut outs formed in the covering between the covered legs of the scaffold.

FIG. 25A is a side view of a vascular occlusion device shown without any cover. In this view, the outer shaft is withdrawn using the slider on the handle to position the distal end of the outer shaft at the proximal end of the scaffold. In this embodiment, in the deployed configuration the outer shaft is withdrawn proximal to the scaffold transition zone with the inner shaft coupler remaining within and covered by the outer shaft.

FIG. 25B is a side view of a vascular occlusion device of FIG. 25A. The slider on the handle is in a proximal position to withdraw the outer shaft or sheath from the scaffold allowing the scaffold to transition from a stowed configuration to deployed configuration as illustrated. In this embodiment, in the deployed configuration the outer shaft is withdrawn proximal to the inner shaft coupler.

FIG. 26A is a side view of a vascular occlusion device in a stowed condition with the outer shaft withdrawn slightly to show the stowed distal end of the scaffold as best seen in the enlarged view of FIG. 26B. The slider on the handle is withdrawn slightly from the distal most position on the handle to only slightly withdraw the outer sheath to the illustrated position. Continued proximal movement of the slider will continue to withdraw the outer shaft or sheath from the scaffold allowing the scaffold to transition from a stowed configuration to deployed configuration.

FIG. 26B is an enlarged view of the distal end of the vascular occlusion device in FIG. 26A.

FIG. 27 is an isometric view of a covered scaffold in a deployed configuration. This scaffold embodiment has three legs to be attached to the inner shaft.

FIG. 28A is a side view of a scaffold in a deployed configuration with a transparent covering. This view shows the covering in relation to the scaffold distal end, along the longitudinal length and into the scaffold transition zone where the pattern of a plurality of cells changes to the legs.

FIG. 28B is a view of the covered scaffold in FIG. 28A where the covering is opaque and the scaffold cell pattern is not visible.

FIG. 29A is a side view of a covered scaffold embodiment having two legs for attachment to the central shaft. This covered scaffold embodiment includes proximal and distal scaffold attachment zones and a central covering portion that is unattached to the scaffold. The covering on the legs to the connection tabs and the distal openings are also seen in this view.

FIG. 29B is a perspective view of the proximal end of the covered scaffold of FIG. 29A. The proximal attachment zone is visible in this view through a distal opening.

FIG. 29C is a perspective view of the distal end of the covered scaffold in FIG. 29A. The proximal attachment zone, the distal attachment zone and the distal openings are visible in this view.

FIG. 30 is a side view of an embodiment of a vascular occlusion device in a deployed condition having a 20% scaffold covering. The slider on the handle is in a proximal position to withdraw the outer shaft or sheath from the scaffold allowing the scaffold to transition from a stowed configuration to deployed configuration as illustrated. The 20% scaffold covering distal end aligns with the scaffold distal end and extends proximally along the longitudinal length of the scaffold to cover approximately 20% of the overall length of the scaffold.

FIG. 31 is a side view of an embodiment of a vascular occlusion device in a deployed condition having a 50% scaffold covering. The slider on the handle is in a proximal position to withdraw the outer shaft or sheath from the scaffold allowing the scaffold to transition from a stowed configuration to deployed configuration as illustrated. The 50% scaffold covering distal end aligns proximal to the scaffold distal end and extends proximally along the longitudinal length of the scaffold to cover approximately 50% of the overall length of the scaffold.

FIG. 32 is a side view of an embodiment of a vascular occlusion device in a deployed condition having an 80% scaffold covering. The slider on the handle is in a proximal position to withdraw the outer shaft or sheath from the scaffold allowing the scaffold to transition from a stowed configuration to deployed configuration as illustrated. The 80% scaffold covering distal end aligns with the scaffold distal end and extends proximally along the longitudinal length of the scaffold to cover approximately 80% of the overall length of the scaffold.

FIG. 33A is a side view of an embodiment of a vascular occlusion device in a deployed condition having an 100% scaffold covering. The 100% scaffold covering distal end aligns with the scaffold distal end and extends proximally along the longitudinal length of the scaffold to cover approximately 100% of the overall length of the scaffold with the exception of a small portion of the end of the device as shown. The slider on the handle is in a proximal position to withdraw the outer shaft or sheath from the scaffold allowing the scaffold to transition from a stowed configuration to deployed configuration as illustrated.

FIG. 33B is a side view of an embodiment of a vascular occlusion device in a deployed condition having an 100% scaffold covering similar to FIG. 33A. The slider on the handle is in a proximal position to withdraw the outer shaft or sheath from the scaffold allowing the scaffold to transition from a stowed configuration to deployed configuration as illustrated. This embodiment illustrates a plurality of openings formed in the proximal end of the covering within the scaffold transition zone. The 100% scaffold covering distal end aligns with the scaffold distal end and extends proximally along the longitudinal length of the scaffold to cover approximately 100% of the overall length of the scaffold.

FIG. 34 is a side view of an embodiment of a vascular occlusion device in a deployed condition having a tapered scaffold covering of a partial cylindrical section. The slider on the handle is in a proximal position to withdraw the outer shaft or sheath from the scaffold allowing the scaffold to transition from a stowed configuration to deployed configuration as illustrated. The tapered scaffold covering distal end aligns with the scaffold distal end and extends proximally along the longitudinal length of the scaffold to various distal positions according to the overall covering shape. In this view the exemplary shaped covering extends over only a few cells of the scaffold in the top portion while covering most all of the cells and nearly reaching the scaffold transition zone in the bottom portion.

FIG. 35 is a perspective view of an embodiment of a vascular occlusion device in a deployed configuration having a scaffold covering extending from the distal end of the scaffold to the scaffold transition zone. The slider on the handle is in a proximal position to withdraw the outer shaft or sheath from the scaffold allowing the scaffold to transition from a stowed configuration to deployed configuration as illustrated. A portion of the distal attachment zone is visible in this view along with a section of the spiral cut inner shaft.

FIG. 36 is a perspective view of an embodiment of a vascular occlusion device in a deployed configuration having a scaffold covering extending from the distal end of the scaffold to the scaffold transition zone for about 270 degrees of the scaffold circumference. A portion of the scaffold along the bottom section remains uncovered as shown. The slider on the handle is in a proximal position to withdraw the outer shaft or sheath from the scaffold allowing the scaffold to transition from a stowed configuration to deployed configuration as illustrated. A portion of the distal attachment zone is visible in this view along with a section of the spiral cut inner shaft.

FIG. 37 is a perspective view of an embodiment of a vascular occlusion device in a deployed configuration having a pair of scaffold covering sections extending from the distal end of the scaffold to the scaffold transition zone for about 45 degrees of the scaffold circumference. A portion of the scaffold along the top and bottom section remains uncovered as shown. The slider on the handle is in a proximal position to withdraw the outer shaft or sheath from the scaffold allowing the scaffold to transition from a stowed configuration to deployed configuration as illustrated. A portion of the distal and proximal attachment zones of one of the scaffold covering sections is visible in this view along with a section of the spiral cut inner shaft.

FIG. 38 is a perspective view of an embodiment of a vascular occlusion device in a stowed configuration. The slider on the handle is in a distal position with the outer shaft or sheath over the covered scaffold and maintaining it in a stowed configuration.

FIG. 39A is an enlarged view of the distal end of the stowed vascular occlusion device of FIG. 38.

FIG. 39B is the enlarged view of FIG. 39A showing the proximal movement of the distal end of the outer shaft or sheath as the slider on the handle advances proximally. The distal end of the covered scaffold and a portion of the distal attachment zone is also shown in this view.

FIG. 39C is the view of FIG. 39B showing the result of continued proximal movement of the slider and corresponding proximal movement of the outer shaft allowing more of the covered scaffold to transition into the deployed configuration.

FIG. 40 is a perspective view of the vascular occlusion device of FIG. 38 after the slider is moved into the proximal position to fully transition the covered scaffold into the deployed configuration. The slider on the handle is in a proximal position with the outer shaft or sheath withdrawn from the covered scaffold which is shown in a deployed configuration.

FIG. 41 is a perspective view of the vascular occlusion device of FIG. 40 with a section of the outer shaft removed to position the deployed covered scaffold adjacent the handle with the slider shown in the proximal position to fully transition the covered scaffold into the deployed configuration as shown.

FIG. 42 is an exploded view of the handle embodiment of FIG. 41.

FIG. 43 is a cross section view of the handle embodiment of FIG. 41.

FIG. 44 is a cross section of a vascular occlusion device positioned for occlusion of the renal arteries and perfusion of the arterial tree in the lower extremities.

FIG. 45 is a flow chart of an exemplary method of providing occlusion with perfusion using an embodiment of a vascular occlusion device according to the method 4500.

FIG. 46 is a flow chart of an exemplary method of providing occlusion with perfusion using an embodiment of a vascular occlusion device according to the method 4600.

FIG. 47 is a flow chart of an exemplary method of providing occlusion with perfusion using an embodiment of a vascular occlusion device according to the method 4700.

FIG. 48 is a side view of an exemplary covered scaffold according to one embodiment of the vascular occlusion device. The covered scaffold indicates the distal attachment zone, the proximal attachment zone and the unattached zone that indicate whether a portion of the scaffold covering is joined to the scaffold structure in that zone.

FIG. 49 is a partial exploded view of a portion of each of the individual layers that together form a multiple layer scaffold covering embodiment. Each one of the layers is shown with an arrow indicating an orientation of a characteristic or quality of that layer. Illustrated orientations are provided relative to the central axis of the scaffold structure as parallel (a), transverse (b) or oblique (c) or (d).

### DETAILED DESCRIPTION

Current treatments/managements for acute kidney injury (AM), especially contrast-induced acute kidney injury are mainly supportive. They include for example, (1) evaluating and stratifying patients with Mehran risk score before performing percutaneous coronary intervention (PCI), (2) avoiding high-osmolar contrast media by using low-osmolar or iso-osmolar contrast media, (3) reducing the amount of contrast media during PCI, and (4) applying intravenously isotonic sodium chloride solution or sodium bicarbonate solution hours before and after PCI, (5) avoiding use of nephrotoxic drugs (such as nonsteroidal anti-inflammatory drugs, aminoglycosides antibiotics, etc.) See Stevens 1999, Schweiger 2007, Solomon 2010. However, none of them were proven with consistent effect in preventing CI-AKI.

Provided herein are devices and systems that specifically focus on solving the two main pathophysiological culprits of CI-AKI, which are renal outer medulla ischemia and/or prolonged transit of contrast media inside the kidneys.

In some embodiments, there are provided a device for treating acute kidney injury (e.g., CI-AKI) comprising a balloon catheter having at least one balloon, at least one sensor associated with the balloon and a position indication means wherein the balloon occludes the orifice of both sides of renal arteries after inflation while allowing blood flow going through the inflated balloon during application of the device inside abdominal aorta. In some embodiments, the position indication means is a radio-opaque marker, or the like.

Radio opaque markers are vital prerequisites on an increasing number of endovascular medical devices and are appropriately provided on the various embodiments to allow positioning of the temporary occlusion device. The value of radio opaque markers is clearly seen in visibility improvement during deployment of the device. Markers allow for improved tracking and positioning of an implantable device during a procedure using fluoroscopy or radiography.

While some embodiments have been described for use in mitigating CI-AKI, alternative non-balloon based occlusion or partial occlusion devices are also provided. Moreover, such alternative partial or complete peripheral occlusion devices simultaneously provide for distal perfusion blood flow into vessels and structures beyond the occlusion device.

As a result, various occlusion device embodiments may be provided that are adapted and configured to provide temporary occlusion of the peripheral vasculature of the suprarenal and infrarenal abdominal aortic area while maintaining distal perfusion.

Exemplary clinical applications include but are not limited to:

Total or nearly total vascular occlusion of blood flow during the surgical treatment of renal tumors through Retroperitoneoscopic Radical Nephrectomy (RRN), Open Radical Nephrectomy (ORN), Open Nephron-sparing Surgery (ONR), or other surgical interventions where it is beneficial to provide temporary vascular occlusion to peripheral organs.

Temporary vascular occlusion of target organs to prevent the influx of solutions (Contrast Medium, Chemotherapy agents) into sensitive organs.

In some embodiments, there is provided a device for treating acute kidney injury, comprising: a balloon catheter having at least one balloon, at least one sensor associated with the balloon and a position indication means wherein the balloon occlude the orifice of both sides of renal arteries after inflation while allowing blood flow goes through the inflated balloon during application of the device inside abdominal aorta.

The various balloon based device descriptions and associated methods may be modified to accomplish any of the above mentioned or other similar vascular occlusion procedures using an embodiment of a partial covered scaffold occlusion device. Additionally, in some embodiments, there is provided for radial expansion of a nitinol scaffold to allow apposition of the attached membrane to the wall of the aorta, to temporarily occlude the flow of blood to the peripheral vasculature. Importantly, embodiments of the radial occlusion device are designed to allow continued distal perfusion while occluding the entrance into the target arteries. In one embodiment, the catheter based radial occlusion system with simultaneous distal perfusion is advanced over a guidewire. In one aspect, a 0.035″ guidewire is used. In some embodiments, proper position of the occlusion device is obtained using one or more radio opaque marker bands or other suitable structures visible to medical imaging systems.

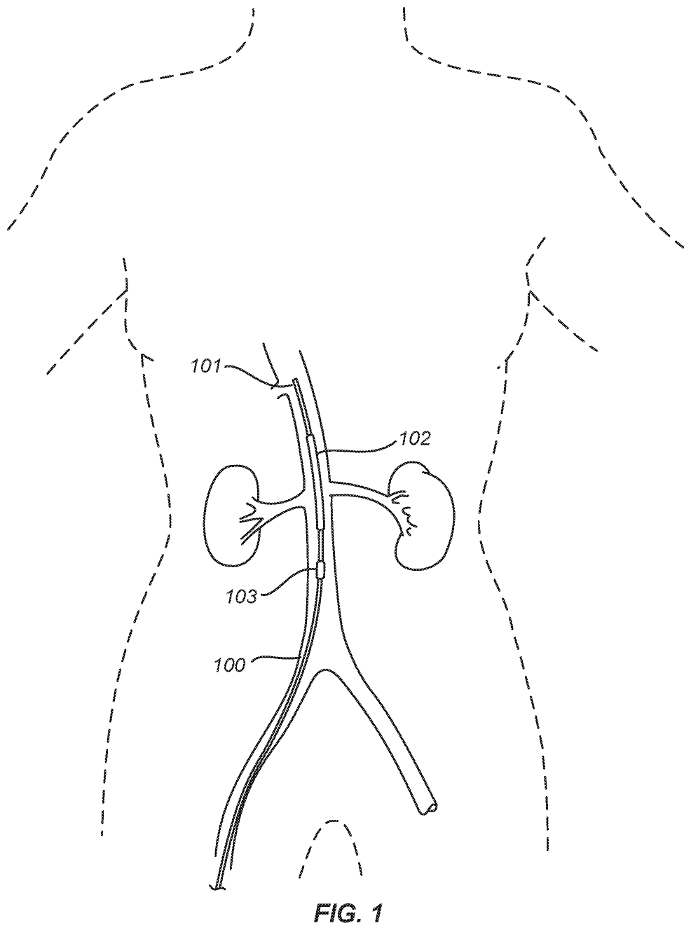

Referring to FIG. 1, an exemplary invention device 100 comprising a balloon catheter 101, a first balloon 102, a second balloon 103 and a radio opaque marker on the tip of the catheter 101 is shown. FIG. 1 shows that the device is inserted via femoral artery and the position of the device is monitored via a radio-opaque marker, or the like. The catheter of the device can be inserted into abdominal aorta by either transfemoral arterial approach or by trans-brachial artery approach or by trans-radial artery approach. The tip with radio-opaque marker is positioned to allow the first balloon at the supra-renal aorta position near orifices of bilateral renal arteries.

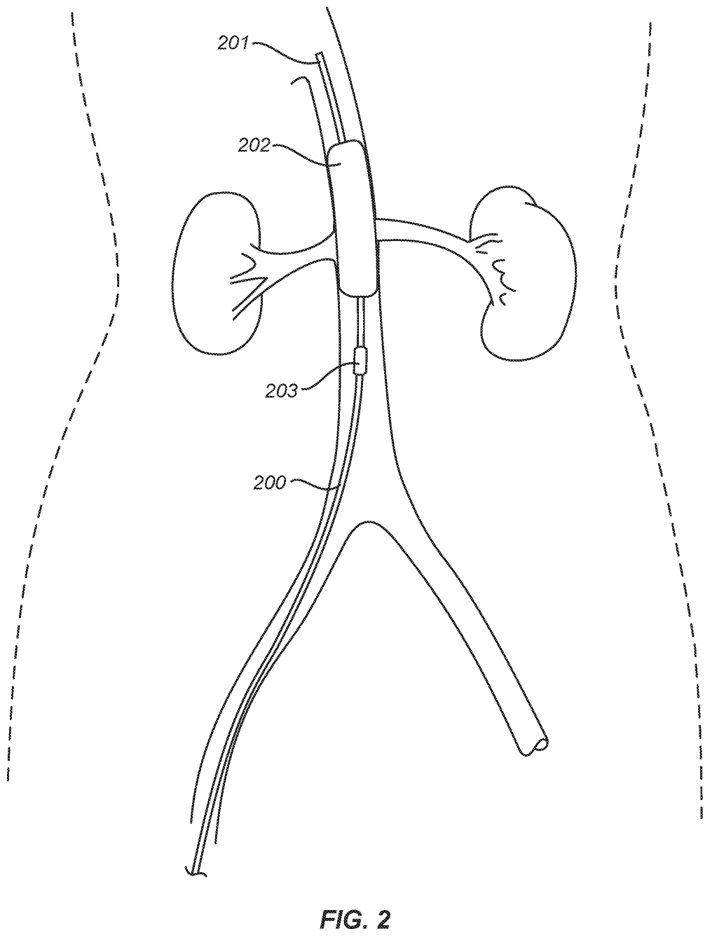

Referring to FIG. 2, a diagram is shown that the device 200 comprising a catheter 201 having a first balloon 202 positioned at the supra-renal aorta position near orifices of bilateral renal arteries and the first balloon 202 is inflated where the inflated first balloon occlude the orifice of both sides of renal arteries so that the bolus influx of contrast media (or any other harmful agents during the application of the invention device) flowing from supra-renal aorta is prevented from entering into renal arteries and cause subsequent toxic effect. The second balloon 203 remains un-inflated.

In certain embodiments, the device comprises a balloon catheter having a first balloon, a second balloon and at least one sensor associated with the second balloon. In some embodiments, the device comprises a balloon catheter having a first balloon, a second balloon and at least one sensor associated with the second balloon.

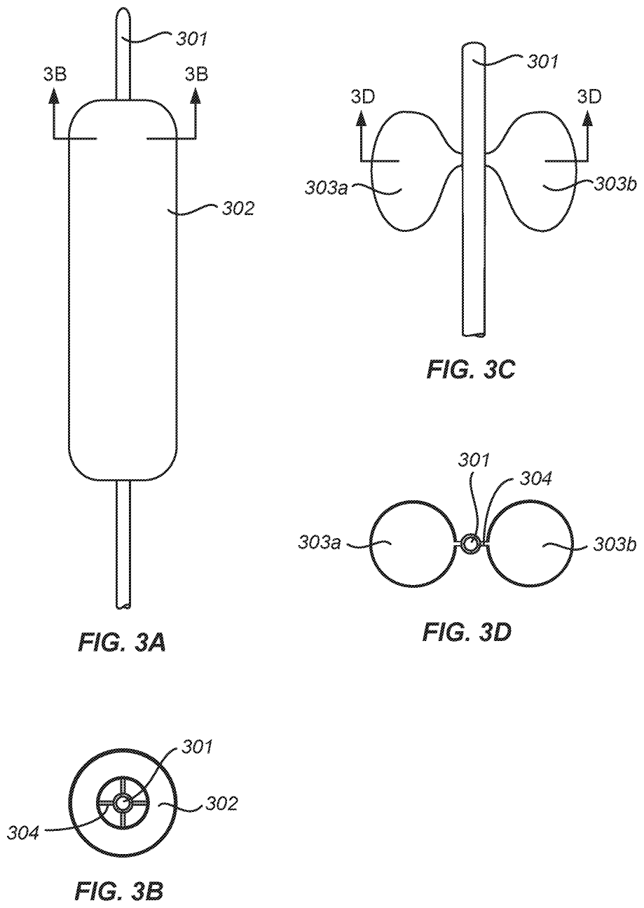

FIGS. 3A to 3D illustrate various embodiments of the first balloon. FIG. 3A shows an inflated first balloon 302 positions along with and circulates the catheter 301. The cross-section view of the inflatable first balloon of FIG. 3A shows a hollow area inside the balloon and outside the catheter 301 (a donut like balloon) allowing blood to flow along the catheter (FIG. 3B). The first balloon 302 is inflated via at least one connection tube 304 from the catheter 301 (four tubes shown in FIG. 3B). FIG. 3C shows other variation of the morphology of inflatable first balloon. A bilateral inflated balloon (303a and 303b) connected to each side of catheter 301 via connection tube 304 to occlude the orifices of both sides of renal arteries are shown in FIG. 3C, which also allows blood to flow along the catheter. FIG. 3D shows the cross-section view of the inflated first balloon of FIG. 3C (a butterfly like balloon). The butterfly like first balloon(s) are connected to the catheter via one or more connection tube 304 (shown one connection tube on each side of the catheter 301). In certain embodiments, the balloon has one, two, three, four or five connection tubes 304 for connection of the first balloon to the catheter and for inflation/deflation means.

In some embodiments, the first balloon is donut-like after inflation. In certain embodiment the first balloon is butterfly-like after inflation.

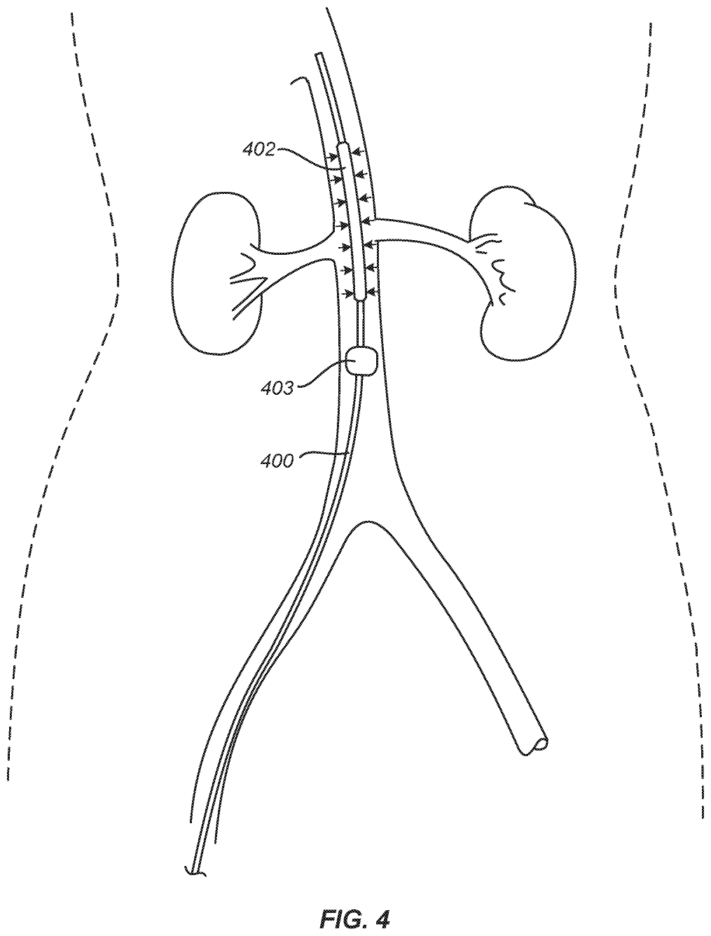

Referring to FIG. 4, it is shown an exemplary device 400 comprising a deflated first balloon 402 after contrast media containing blood passed by and then the second balloon 403 is inflated at the location of infra-renal aorta near the orifice of renal arteries.

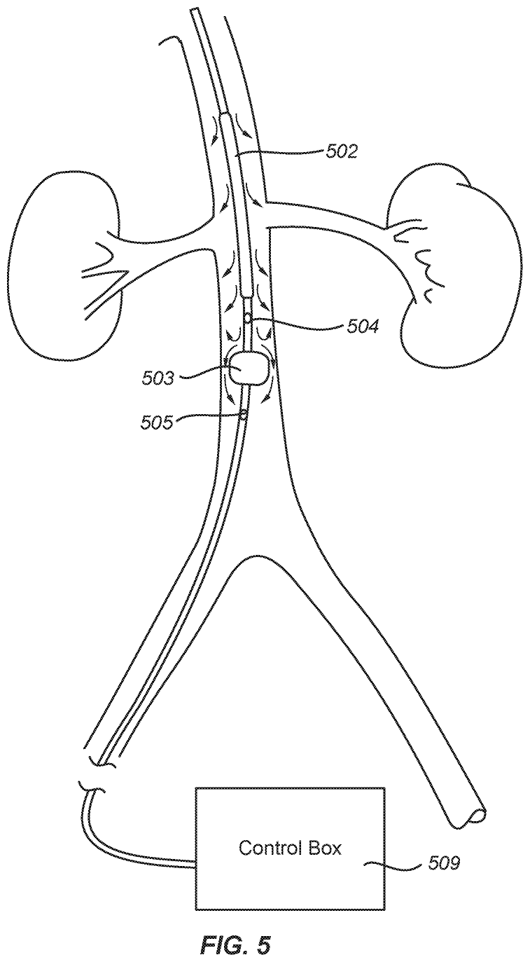

The inflation of the second balloon 503 is to the extent not totally occludes the aorta blood flow. As shown in FIG. 5, in the aorta, the vortex blood flow caused by the inflated second balloon distension will facilitate (augment) renal artery blood flow. In some embodiments, there is at least one sensor associated with the first balloon or second balloon for the control of inflation/deflation of either the first and/or second balloon. In some embodiments, the sensor is a pressure sensor. In some embodiments, the sensor is a size measuring sensor related to the size of either the first balloon or the second balloon. As shown in FIG. 5 as a non-limited example, there are one pressure sensor 504 at lower side of the first balloon (or at the upper side of the second balloon) and another pressure sensor 505 at lower side of the second balloon.

The analysis of data from the pressure sensors can be used as instantaneous titration of distention degree of the second balloon to provide adequate pressure gradient, and hence adequate vortex flow into renal arteries. In addition, the altered aorta blood flow will increase the renal artery blood flow, due to the location proximity and the diameter of the distended the second balloon. In some embodiments, the diameter of the distended second balloon is adjustable such that the diameter of the distended balloon is not too large to totally obstruct aorta blood flow and the altered aorta blood flow will not cause inadequacy of aorta blood flow at distal aorta or branches of aorta, i.e. right and left common iliac artery. Furthermore, the aorta wall will not be injured by the balloon distension.

Also shown in FIG. 5, there is a control box 509 outside the patient body, in connection with the balloon catheter. The control box will serve several functions: inflation and deflation of the first and second balloons, pressure sensing and/or measurement of upper and lower pressure sensors, normal saline titration via an included infusion pump with titratable infusion rate.

In some embodiments, there are two sets of pressure sensors, one at the supra-renal aorta side of the balloon, the other at the infra-renal aorta side of the balloon. The two sensors can continuously measure the pressure and the measured data can be exhibited at the control box outside of the patient's body. The pressure difference between the two sensors will be exhibited on the control box. Physicians can read the pressure difference and adjust the size of balloon by way of a control box. Or the control box can do the adjustment of size of balloon automatically.

In some embodiments, the device for treating acute kidney injury further comprises a side aperture on the balloon catheter for application of normal saline or other medication infused from the control box, through the catheter into the supra-renal aorta. In some embodiments, normal saline (or other medication) is applied via a side aperture between the first and second balloon. In some embodiments, normal saline (or other medication) is applied via the tip of catheter.

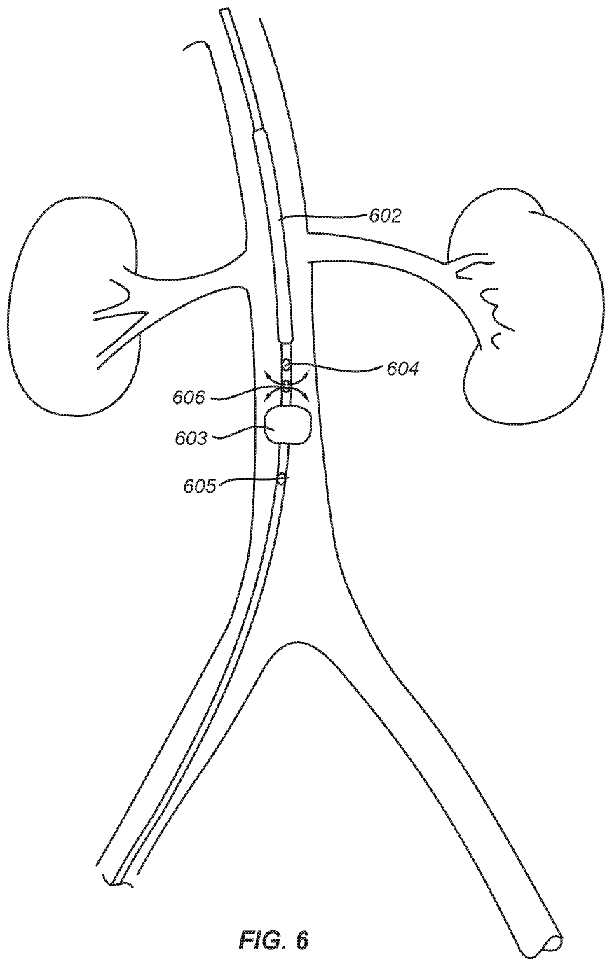

As illustrated in FIG. 6, an exemplary device for treating AKI comprising a first balloon 602, a second balloon 603 (shown inflated), a first sensor 604, a second sensor 605 and a side aperture 606 where normal saline can be infused into the supra-renal aorta via the side aperture 606. By infusion of normal saline into the supra-renal aorta, the renal artery blood flow can be further augmented. Furthermore, it avoids the direct fluid overload burden onto the heart, especially when patients already have congestive heart failure. For the treatment of CI-AKI, the infusion of normal saline into the supra-renal aorta also dilutes the concentration of contrast media in the supra-renal aorta, therefore reduces the concentration of contrast media and thus reduce the adverse effect of hyperviscosity caused by contrast media to the kidneys, after the contrast media flowing into the kidneys. In some embodiments, the infusion rate of normal saline through the side aperture into aorta can be controlled by the control box. In some embodiments, there is a control pump inside the control box to apply normal saline via the side aperture. In some embodiments, the control pump is in a separate unit. In some embodiments, the medication is a vasodilatory agent. In certain embodiments, the vasodilatory agent is Fenoldopam, or the like. In certain embodiments, the medication such as Fenoldopam, or the like is infused via the side aperture for prevention and/or treatment of CI-AKI.

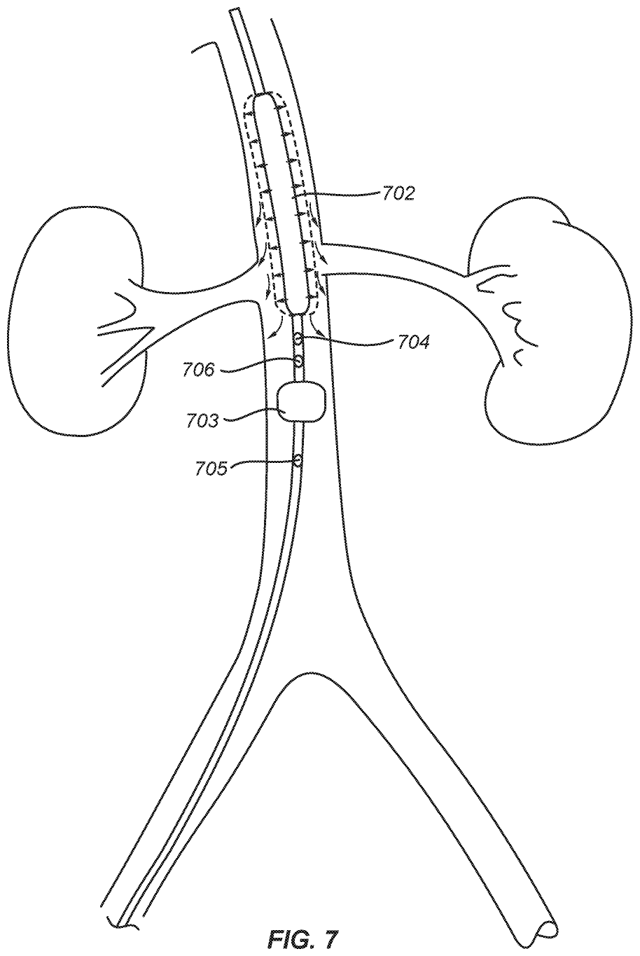

FIG. 7 demonstrates another variation of the invention device comprising a balloon catheter having a first balloon 702, a second balloon 703 (shown inflated), at least one sensor (shown two sensors 704 and 705) and a side aperture where the first balloon 702 can exert renal artery blood flow augmentation by periodic inflation and deflation. As shown in FIG. 7, when the first balloon is inflated, it will not be inflated to totally occlude the orifice of renal arteries as shown in FIG. 2. Such periodic balloon inflation/deflation will cause blood flow into renal arteries.

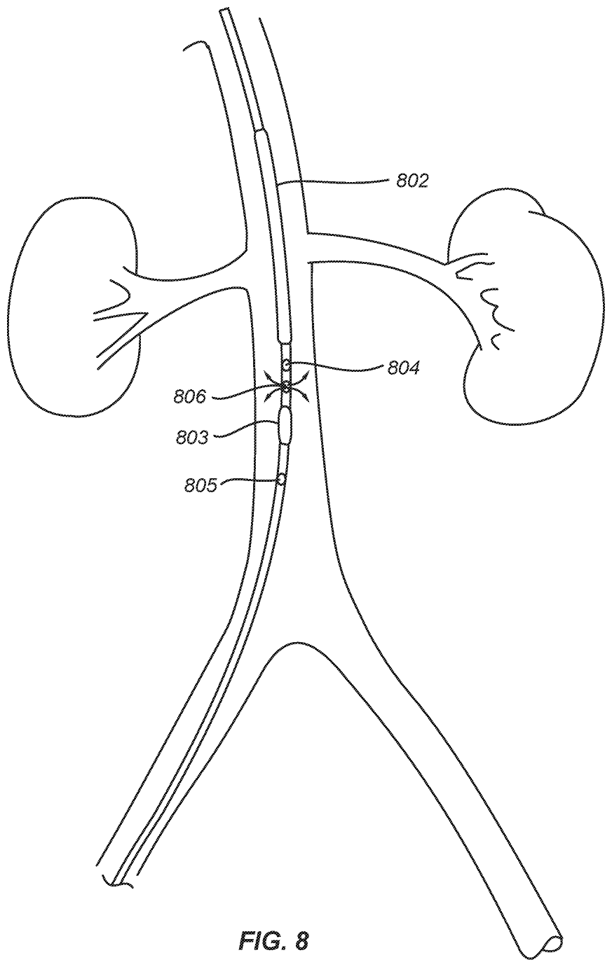

Referring to FIG. 8 at the end of percutaneous coronary intervention (PCI), both the first and second balloons will be deflated and either removed or remained inside aorta and normal saline will be continuously infused via a side aperture 806 as postprocedural hydration.

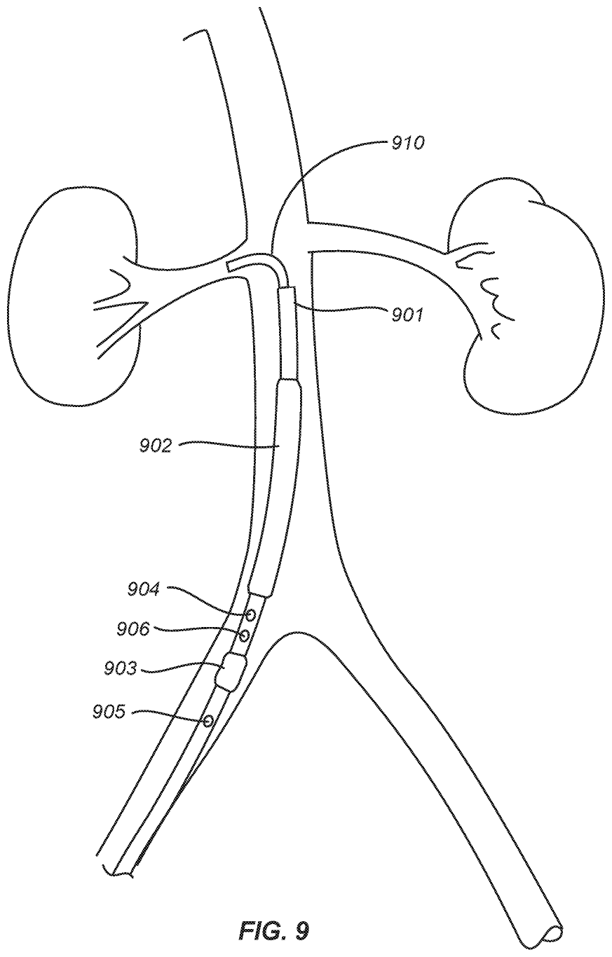

As illustrated in FIG. 9, an exemplary device for treating AKI comprising a catheter 901, a first balloon 902, a second balloon 903, a first sensor 904, a second sensor 905, a side aperture 906 further includes a guidewire 910. The guidewire is inserted into renal artery via a catheter. When guidewire is inside renal artery, the outer sheath catheter is also inserted into renal artery.

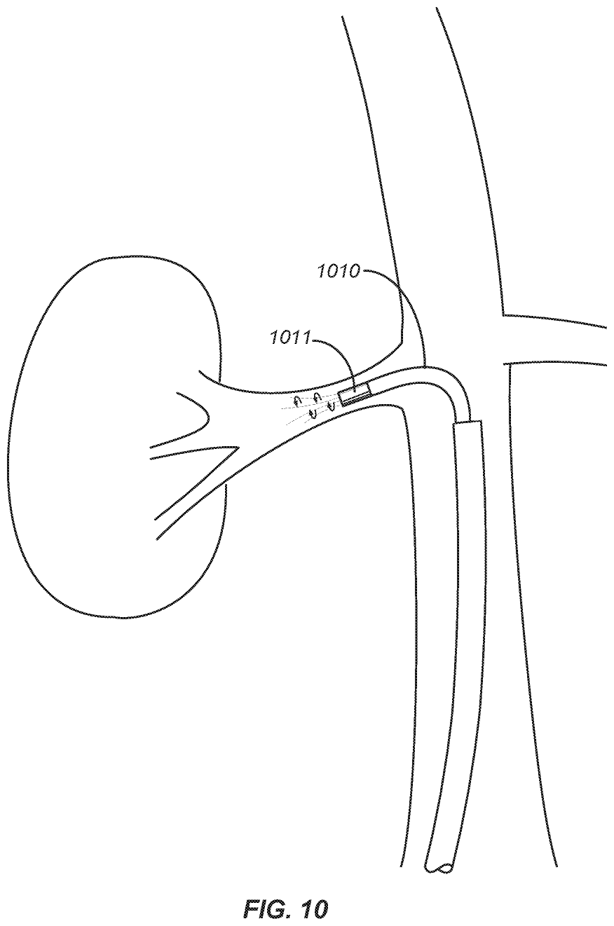

FIG. 10 shows that a spinning propeller 1011 is inserted from outer sheath catheter into renal artery through the guidewire 1010. The exemplary unidirectional flow pump such as a spinning propeller then spins around the central guidewire and generate directional augmented renal artery blood flow toward the kidney, hence achieves the goal of augmented renal artery flow.

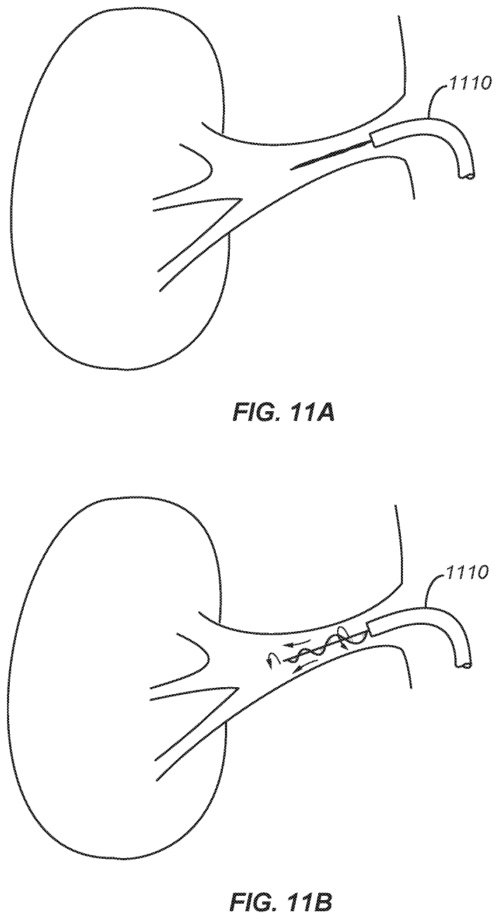

FIGS. 11A and 11B show variations of the spinning propeller. The spinning propeller in some embodiments is wing shape, fin shape, or the like.

In some embodiments, the balloon catheter further includes a guidewire and a spinning propeller. In certain embodiments, the spinning propeller spins around the central guidewire to generate directional augmented renal artery blood flow toward the kidney. In certain embodiments, the spinning propeller is wing shape or fin shape. In certain embodiments, the device further comprises another catheter comprising a guidewire and a spinning propeller to generate directional augmented blood flow to the other kidney. In certain embodiments, the additional catheter having a spinning propeller is functioned independently and simultaneously with the balloon catheter to generate directional augmented blood flow to each side of kidney.

In some embodiments, the infra-renal side of the vascular occlusion device or the disturbing means (such as infra-renal tunnel membrane) can inject saline via injection hole or using the inner shaft into the aorta to dilute the contrast media before it flows into the renal arteries. One or more injection holes may be located along the inner shaft proximal to the atraumatic tip or proximal or distal to the inner shaft coupler 1530.

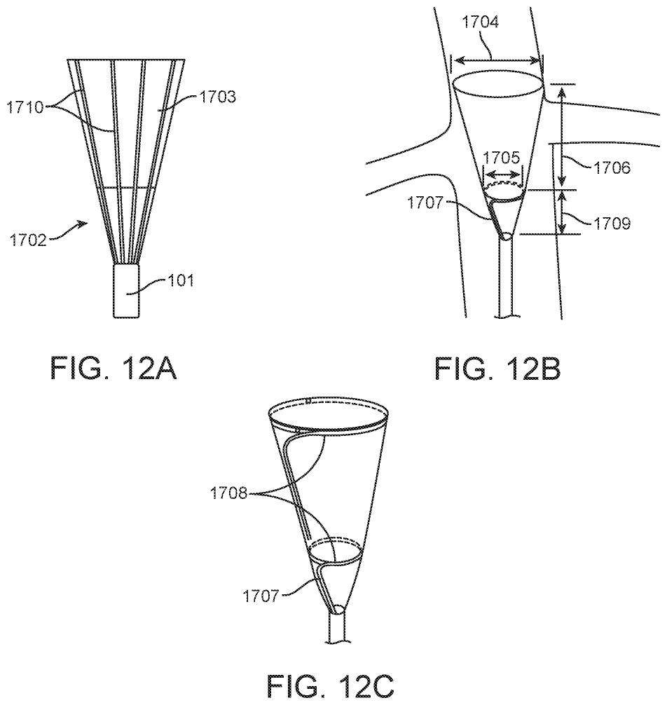

As illustrated in FIG. 12A, which provides yet another embodiment of the flow disturbing means, is a cone shaped wire device 1702 partially covered with tunnel membrane 1703 which is deployed from catheter 1701. FIG. 12B provides an exemplary specification of the cone shaped wire device 1702 of FIG. 12A where the diameter of the distal opening 1704 is about 3 to 3.2 cm or about 3.0 cm. Thus the outer rim of the wire device 1702 is either tightly fitted inside the aorta (of e.g., 3.0 to 3.2 cm diameter) or loosely situated with little space allowing blood seeping through. The diameter of the distal opening 1704 is based on various diameters of an aorta (typically from about 5 cm to about 2 cm) in the patients where the device is deployed. In some embodiments, the distal opening has a diameter of about 5 cm to about 1.5 cm; in some embodiments, the distal opening has a diameter of about 4.5 cm to about 1.7 cm; in some embodiments, the distal opening has a diameter of about 4 cm to about 1.8 cm; about 3.5 cm to about 1.8 cm; or about 3 cm to about 2.0 cm. A tunnel membrane 1703 is covered from the edge of the distal opening 1704 to the proximal opening 1705 of the wire device. The height (1706, see FIG. 12B, where is the distance of blood flowing through) of the tunnel membrane in some embodiments is about 1.5 cm to about 4 cm, about 2 cm to about 3.5 cm, about 2.5 cm to about 3.0 cm (as shown in FIG. 11B is 3 cm). In some embodiments, the height 1706 of the tunnel membrane is about 2 cm, about 3 cm, or about 4 cm. The proximal opening 1705 allows the blood flow through with restricted speed that creates a disturbing of blood flow allowing that the renal arteries intakes blood flow from the infra-renal aorta, where the contrast media has been diluted by the blood flow. To create such an effective blood flow disturbing caused by a disturbing means (e.g, the device 1702), in some embodiments, the diameter of the proximal opening is about one-fourth to about three-fourth of the diameter of the distal opening. In some embodiments, the diameter of the proximal opening is about one-third of the diameter of the distal opening. For example, as shown in FIG. 12B the diameter of the bottom opening 1705 is about 1.0 cm. Relative to where the blood flowing through from the proximal opening, blood releasing height 1709 is designed to be about one-half to about three times of the diameter of the proximal opening. The ratio relationship between blood releasing height 1709 and proximal opening 1705 is based on (1) how the wire device restricts blood flow which creates disturbance, (2) the structural strength of the wire device, and (3) the diameter relationship between the distal opening and the proximal opening.

To support such cone shaped structure, the wire device comprises wires 1710 with at least 3 wires. In some embodiments, there are 4 to 24 wires, 5 to 22 wires, 6 to 20 wires, 8 to 18 wires, or 10 to 16 wires. In some embodiments, there are 3, 4, 5, 6, 7, 8, 9, 10, 11, 12, 13, 14, 15, 16, 17, 18, 19, or 20 wires in the wire device partially covered with tunnel membrane. If needed, a skilled person in the art can prepare a wire device in accordance with the practice of the present invention to any number of wires suitable to provide a disturbing means. The wire may be any superelastic material such as nitinol.

Pseudoelasticity, sometimes called superelasticity, is an elastic (reversible) response to an applied stress, caused by a phase transformation between the austenitic and martensitic phases of a crystal. It is exhibited in shape-memory alloys. Pseudoelasticity is from the reversible motion of domain boundaries during the phase transformation, rather than just bond stretching or the introduction of defects in the crystal lattice (thus it is not true superelasticity but rather pseudoelasticity). Even if the domain boundaries do become pinned, they may be reversed through heating. Thus, a superelastic material may return to its previous shape (hence, shape memory) after the removal of even relatively high applied strains.

The shape memory effect was first observed in AuCd in 1951 and since then it has been observed in numerous other alloy systems. However, only the NiTi alloys and some copper-based alloys have so far been used commercially.

For example, Copper-Zinc-Aluminum (CuZnAl) was the first copper based superelastic material to be commercially exploited and the alloys typically contain 15-30 wt % Zn and 3-7 wt % Al. The Copper-Aluminum, a binary alloy, has a very high transformation temperature and a third element nickel is usually added to produce Copper-Aluminum-Nickel (CuAlNi). Nickel-Titanium Alloys are commercially available as superelastic material such as nitinol. In some embodiments, the superelastic material comprises copper, aluminum, nickel or titanium. In certain embodiments, the superelastic material comprises nickel or titanium, or combination thereof. In certain embodiments, the superelastic material is nitinol.

Specific structures can be formed by routing wires (bending one or a few wires and weaving into final shape) or cutting superelastic tube (laser cutting out the unwanted parts and leaving final wires in place) or cutting superelastic sheet (laser cutting out the unwanted parts and annealing the sheet into a cone shape.

Similarly, in some embodiments, the disturbing means (e.g., the wire device 1702) can inject saline from one or more injection hole 1708 via an infusion tube 1707 at the distal opening 1704 or the proximal opening 1705, or combination thereof into the aorta to dilute the contrast media further before it flows into the renal arteries. See FIG. 12C. In some embodiments, the injection hole(s) is on the catheter, for example at the position close to the tip of the catheter where the disturbing means is deployed.

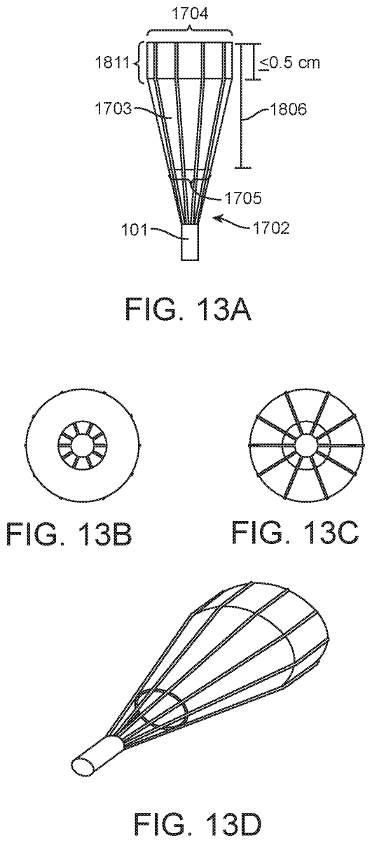

In some embodiments, the cone shaped wire device comprises an upper cylinder portion 1811 as illustrated in FIG. 13A. The upper cylinder portion 1811 is used to form tight contact of the device on the aorta wall. This tight contact supports the device against high pressure due to high blood flow rate. This tight contact prevents contrast media from leaking through the contact interface (without blood seeping through). To avoid occlusion of arteries branching from supra-renal aorta by upper cylinder portion, which is about 0.5 cm apart, the height of the upper cylinder portion should not be more than 0.5 cm to avoid blocking artery branches. The height 1806 of the distal opening to the proximal opening should be about 1.5 cm to about 4 cm, about 2 cm to about 3.5 cm, or about 2.5 cm to about 3.0 cm.

As illustrated in FIG. 13A (a side view), which provides yet a variation of the embodiment of FIGS. 12A-12C, a cone-cylinder shaped wire device 1802 partially covered with a coating, sheet or tunnel membrane 1803 from the rim of the distal opening 1804 to proximal opening of 1805, which is deployed from catheter 1801. FIG. 13B shows a top view of the wire device 1802. FIG. 13C shows a bottom view of the wire device 1802. FIG. 13D provide an isometric view of the wire device 1802.

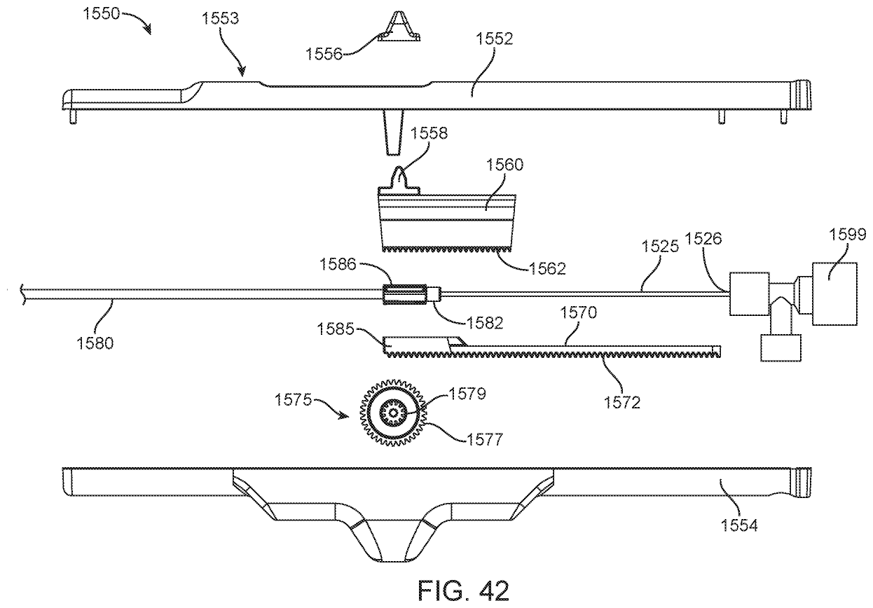

In yet another embodiment, first and second balloons 102, 103 may be replaced by an expanded foam or other biocompatible sealant structure that may be compressed against the vessel wall. The deployed sealant structure under radial force generated by the wire structure or other scaffold embodiment seals against the vessel wall sufficient to fully or at least substantially seal to the vessel wall such that all or substantially all of the blood flow within the vessel flows through the tunnel membrane. Additionally or optionally, the tunnel membrane may be solid or include apertures to allow for various amounts of localized perfusion (see for example FIGS. 42-47). In yet another aspect, balloons 102 and 103 are replaced by a sleeve. The sleeve may be formed from an ePTFE or other compressible biocompatible material. In yet another aspect, the proximal and distal structures about the tunnel membrane may be coated wires, or a hydrogel. In still further alternative structures, one or more of the wires 107 may extend to the ends of the structure, or optionally include a zig-zag pattern and formed from nitinol for self-expansion. It is to be appreciated that in some embodiments, no balloons are utilized but the amount of sealing for a particular embodiment is provided by an alternative radial force sealing structure as describe herein.

The position indication means 105 may for example be a radio-opaque marker. One or more position indication means 105 may be located on the tip of the catheter 101, on the proximal balloon 103, on the distal balloon 102, or any combination thereof. The position indication means 105 may be used to monitor the position of the device 100 upon insertion, during use, and during removal. The device 100 may be inserted into the abdominal aorta for example by using either a trans-femoral arterial approach, a trans-brachial artery approach, or a trans-radial artery approach.

In some embodiments, the aperture 106 and the surrounding wire 107 comprise at least one set of the aperture 106 and the surrounding wire 107 on the tunnel membrane. In some embodiments, there are one to four sets, two to six sets, three to nine sets, four to twelve sets, five to fifteen sets, or six to eighteen sets. In some embodiments, there may be 1, 2, 3, 4, 5, 6, 7, 8, 9, 10, 11, 12, 13, 14, 15, 16, 17, or 18 sets of the aperture and the surrounding wire on the tunnel membrane. If needed, a person skilled in the art can prepare a wire device in accordance with the practice of the present disclosure to any number sets of the aperture and the surrounding wire suitable to provide a flow passage means. The wire may be any superelastic material, for example nitinol. The wire may be made of any superelastic or pseudoelastic material, for example nitinol, alloys of nickel-titanium, or any combination thereof. In some embodiments, the superelastic material may comprise one or more of nickel, titanium, or any combination thereof. Alternatively, any of the above may be modified for use as a wire frame scaffold used with a covering, membrane, coating or tunnel membrane described herein without provision for an aperture 106. Additionally or optionally, the braid embodiments described herein may be include interleaved longitudinal wires to provide an adjustable stiffness. Additionally, the longitudinal wires are provided so as to remain aligned to the central axis of the catheter. Still further, aspects of the fabrication technique and weave patterns used in the braid structure are utilized to modify or adjust a foreshortening characteristic of the braid structure when used as an partially covered scaffold vascular occlusion device.

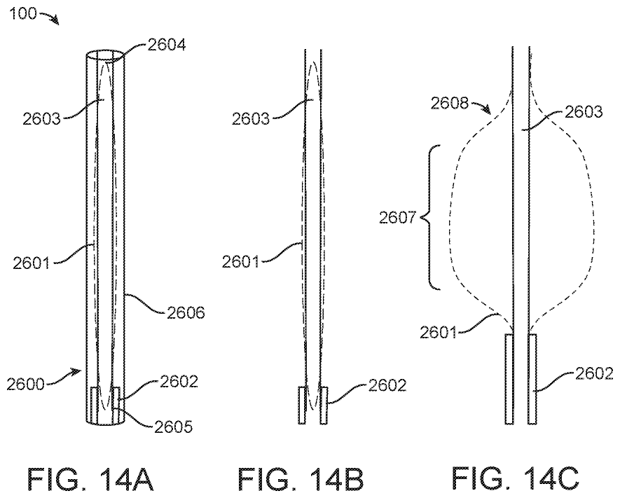

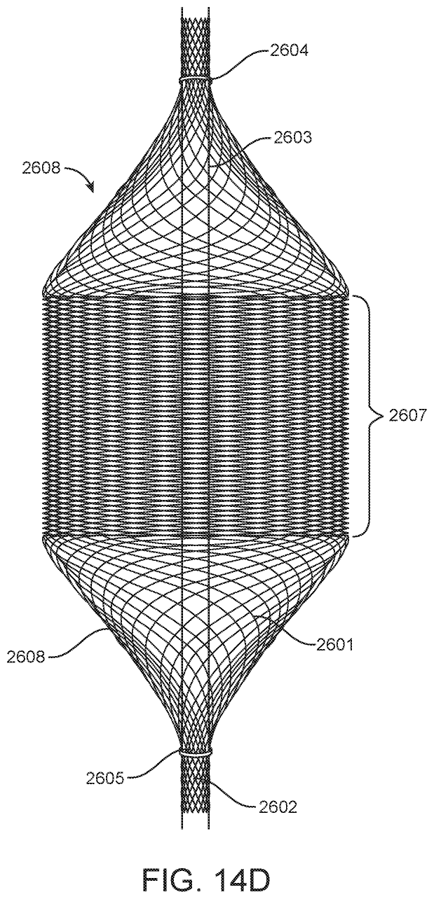

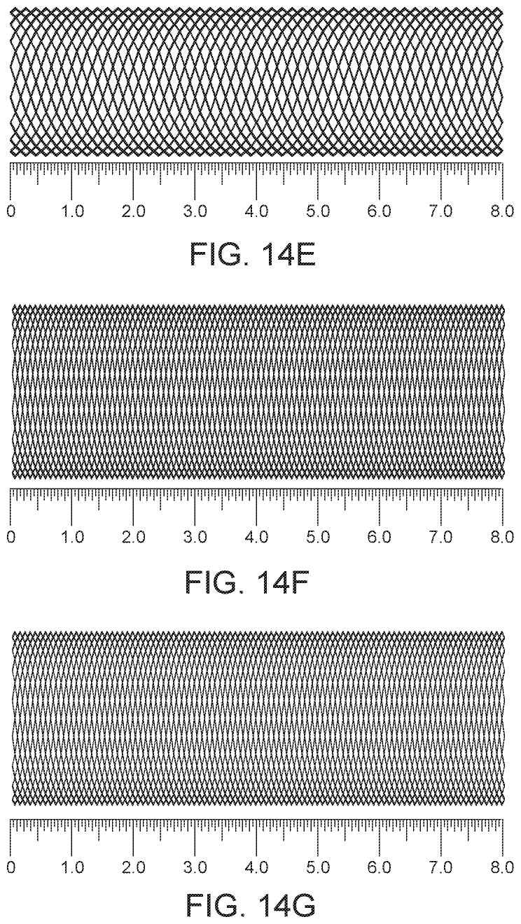

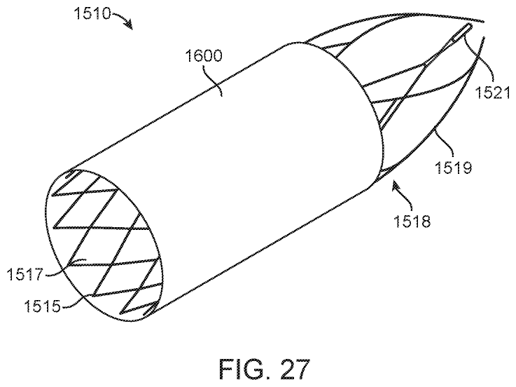

FIGS. 14A-14G show yet another embodiment of the present disclosure. The catheter device 100 may comprise a catheter shaft 2600 actuated to deploy an occlusive element 2601 to occlude the renal artery openings. The occlusive element 2601 may, for example, be an expandable mesh braid. In additional embodiments, the mesh braid is at least partially covered by a covering, membrane, coating or tunnel membrane described to enhance the ability to provide complete or partial occlusion with distal profusion. The covering is omitted from the various views so as not to obscure details of the braid structures. The cover, coating, membrane or tunnel membrane may be a full covering of the underlying structure or scaffold including a partial, single or multiple layer scaffold covering implemented as shown in FIGS. 27, 28B, 29A-29C, 30, 31, 32, 33A, 33B, 34, 35, 36, 37, 39C, 40 and 41. In other aspects where the scaffold is formed from an expandable mesh braid, this structure may comprise a tubular, metal mesh braid comprising a plurality of mesh filaments. The expandable mesh braid may comprise a shape-memory material such as Nitinol and may be biased to be in the expanded configuration. The device may further comprise a position indication features, for example, at least a portion of the catheter device may be radio-opaque. In one aspect, the atraumatic tip 1532 of the inner shaft 1525 is radio-opaque.

The expandable mesh braid or the scaffold may for example be made of a superelastic material such as nitinol. The braid or scaffold may be made of any superelastic or pseudoelastic material, for example nitinol, alloys of nickel-titanium, or any combination thereof. In some embodiments, the superelastic material may comprise one or more of copper, aluminum, nickel, titanium, or any combination thereof. The expandable mesh braid may for example be made of steel or any other mesh-grade material. The expandable mesh braid may be provided with a tunnel or occlusion membrane 1600 embodiment as described herein. Optionally, the braid or scaffold or portion thereof may be coated such as with a hydrophobic coating, a hydrophilic coating, or a tacky coating for enhanced occlusion properties. Additionally or optionally, one or both of the inner and outer braid surfaces may be coated with ePTFE, PTFE, polyurethane or silicone. In some embodiments, the thickness of the coating is from 5 to 100 microns. Still further, the shape of the braid or scaffold may be adjusted to better fit into the geometry of the abdominal aorta, for example the diameter of the lower part of the braid may be smaller than the diameter of the upper part of the braid. It is to be appreciated that these coating concepts may also be applied to the various scaffold embodiments described herein.

FIG. 14A shows a catheter shaft 2600 comprising an outer shaft 2602 and an inner shaft 2603 disposed therein which are translatable relative to one another. The distal end 2604 of the expandable mesh braid 2601 may be coupled to the inner shaft 2603 while the proximal end 2605 of the expandable mesh braid 2601 may be coupled to the outer shaft 2602 such that translation of the inner shaft 2603 relative to the outer shaft 2602 deploys or collapses the expandable mesh braid 2601. The catheter shaft 2600 may further comprise a cover 2606 to protect the catheter shaft device 100 during insertion into the abdominal aorta. The cover 2606 may be removed upon positioning the catheter shaft device 2600 at a desired location.

FIG. 14B shows the catheter shaft device 100 with expandable mesh braid 2601 coupled to the inner 2603 and outer 2602 shafts. The expandable mesh braid 2601 is shown in a low-profile configuration which may be used for delivery of the device 100 through the vasculature prior to deployment. The low-profile configuration may be axially elongated and radially collapsed.

FIG. 14C shows the catheter shaft device 100 following actuation of the inner shaft 2603 relative to the outer shaft 2602 for deployment of the expandable mesh braid 2601. The expandable mesh braid 2601 is shown in an expanded configuration such that the device 100 occludes the renal artery ostia (also referred to herein as orifices) to prevent contrast agent from flowing into the renal arteries of a patient when a bolus of the contrast agent has been introduced into the vasculature. The expanded configuration may be axially foreshortened and radially expanded. In the expanded configuration, the expandable mesh braid 2601 may comprise a minimally porous portion 2607, for example a high-density mesh brain filament portion. The minimally porous portion 2607 may be a region where the braid 2601 is axially foreshortened to increase filament density. The expandable mesh braid 2601 in the expanded configuration may comprise one or more porous end portions 2608 adjacent to the minimally porous portion 2607 so as to allow blood to flow through the braid 2601 from the supra-renal aorta to the infra-renal aorta, bypassing the occluded renal arteries. The one or more porous end portions 2607 may comprise low mesh braid filament density portions.

Actuation of the catheter shaft for deployment of the expandable mesh braid may, for example, comprise translating the inner and outer shafts such that the distal end of the outer shaft moves closer to the distal end of the inner shaft.

FIG. 14D shows a prototype of a catheter shaft device 2600 with expandable mesh braid 2601. The embodiment comprises a tubular metal mesh braid 2601 comprising a plurality of mesh filaments made of Nitinol, an outer shaft 2602, and an inner shaft 2603. The distal end 2604 of the expandable mesh braid 2601 is coupled to the inner shaft 2603 while the proximal end 2605 of the expandable mesh braid 2601 is coupled to the outer shaft 2602. Translation of the inner shaft 2603 relative to the outer shaft 2602 deploys or collapses the expandable mesh braid along with any attached coating, covering, or membrane. In its expanded configuration, the expandable mesh braid 2601 comprises a minimally porous portion 2607 with which to occlude the orifices of the renal arteries. The expandable mesh braid further comprises two porous end portions 2608 which may allow blood to flow through the braid 2601 from the supra-renal aorta to the infra-renal aorta, bypassing the occluded renal arteries. FIG. 14E shows the expandable mesh braid 2601 with fully open mesh. FIG. 14F shows the expandable mesh braid 2601 with a partially collapsed mesh. FIG. 14G shows the expandable mesh braid 2601 with fully collapsed mesh.

The vascular occlusion device 1500 may further comprise a time-delayed release mechanism configured to automatically collapse the expandable occlusion structure (ie., mesh braid or scaffold) after a pre-determined amount of time following deployment. The time-delayed release mechanism may, for example, comprise an energy accumulation and storage component and a time-delay component. For example, the time-delayed release mechanism may comprise a spring with a frictional damper, an example of which may be included in the handle 1550. The energy accumulation and storage component may for example be a spring or spring-coil or the like. The time-delayed release mechanism may for example be adjustable by one or more of the user, the manufacturer, or both. The time-delayed release mechanism may further comprise a synchronization component to synchronize the injection of a contrast media or other harmful agent with the transition of the vascular occlusion device between a stowed configuration and a deployed configuration to aid in preventing harm to structures vascularized by the peripheral vessels that are subject to selective occlusion by operation of the device. For example, injection of contract may be synchronized with occlusion of the renal arteries by the expandable mesh braid or covered scaffold such that a contrast media may be prevented or substantially prevented or greatly reduced amounts from entering the renal arteries.

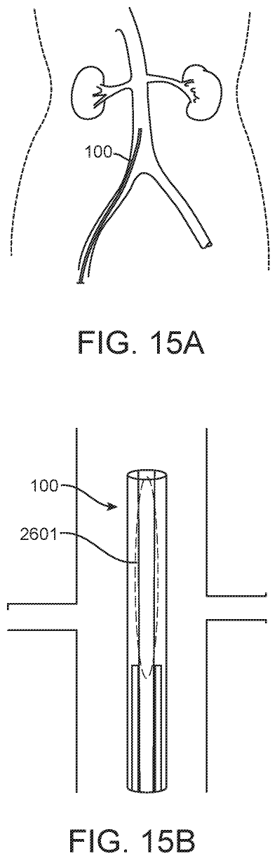

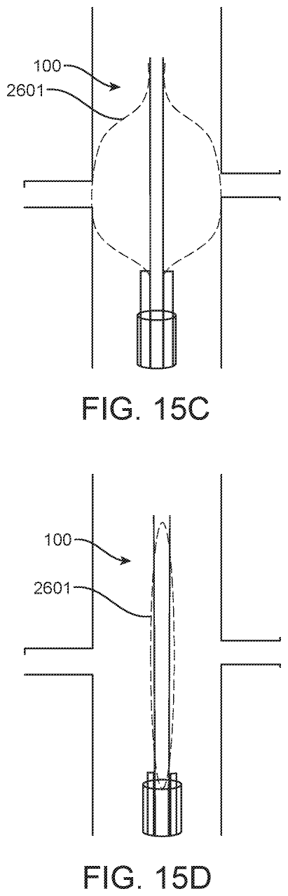

FIGS. 15A-15D show the deployment of the embodiment of FIGS. 14A-14G. Similar deployment steps may be used for all of the embodiments described herein. As shown in FIG. 15A, the device 100 may be inserted into the abdominal aorta via the femoral artery. Alternatively, the device 100 may be inserted into the abdominal aorta via the branchial or radial arteries. As shown in FIG. 15B, the device 100 may be guided to a desired location within the abdominal aorta by monitoring a position indication means, for example a radio-opaque marker or a radio-opaque portion of the catheter. The device 100 may for example be positioned such that deployment of the expandable mesh braid 2601 occludes the orifices of the renal arteries. FIG. 15C shows the expandable mesh braid 2601 deployed at a desired position so as to occlude the orifices of the renal arteries. The expandable mesh braid 2601 may be deployed prior to or simultaneously with injection of a contrast agent into the abdominal aorta of a patient so as to prevent the contrast agent from entering the renal arteries. After the bolus of contrast agent has been introduced, the expandable mesh braid 2601 may be collapsed to allow blood flow to the renal arteries to resume, as shown in FIG. 15D.

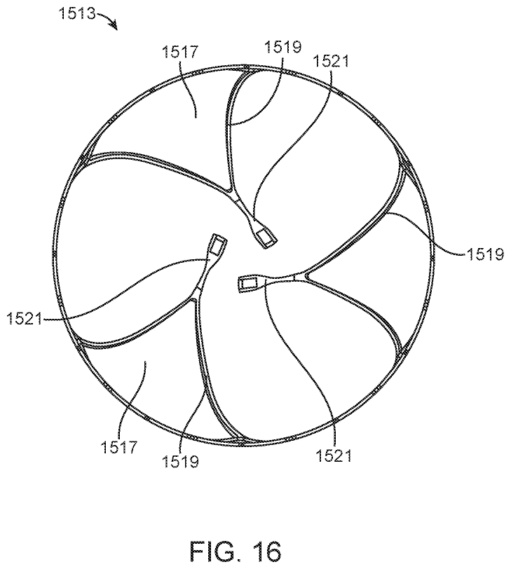

Various embodiments of a vascular occlusion device 1500 are described and illustrated herein and with specific reference to FIGS. 16-49. In general, these embodiments along with those detailed in FIGS. 1-15, relate to a vascular occlusion device configured with structure (e.g., a scaffold structure in relation to FIGS. 16-49) that is adapted to provide selective occlusion with perfusion when appropriately positioned within the vasculature. An exemplary vascular occlusion device 1500 includes a handle 1550, an outer shaft 1580, an inner shaft or hypotube 1525 and a covered scaffold coupled to the distal end of the inner shaft 1525. A slider 1556 on the handle 1550 is coupled to the outer shaft 1580. As the slider 1556 moves along a slot 1553 in the handle, the outer shaft moves relative to the scaffold 1510 allowing the scaffold to move into a deployed configuration or remain within a stowed configuration.

The scaffold 1510 includes a central longitudinal axis 1511 along the inner shaft 1525. The scaffold 1510 includes a proximal end 1513, a distal end 1515, and a plurality of cells 1517. There is also a scaffold transition zone 1518 adjacent to the two or more legs 1519. Each leg 1519 terminates on proximal end in a connection tab 1521. Inner shaft coupler 1530 with key features 1531 to mate with connection tabs 1521 on the proximal end of legs 1519.

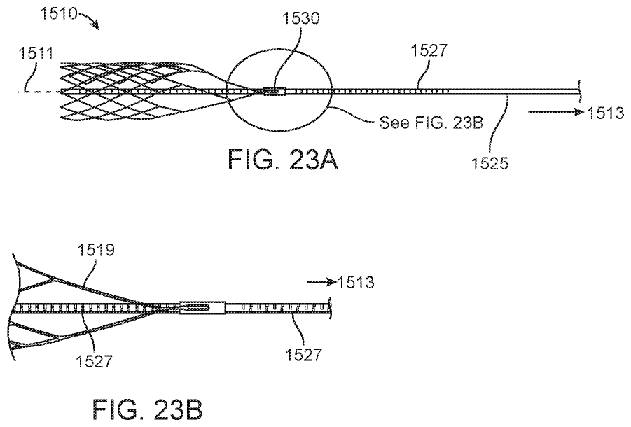

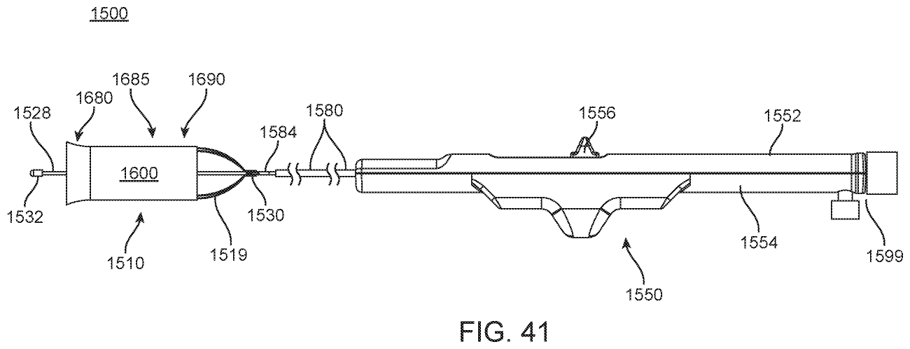

The inner shaft 1525 has a proximal end 1526 and a distal end 1528. The proximal end 1526 is in communication with the hemostasis valve 1599 in the proximal end of handle 1550. (See FIGS. 41, 42 and 43). The distal most end of the inner shaft 1525 has an atraumatic tip 1532. The inner shaft may be a hypotube suited to provide access to a guidewire via the inner shaft lumen. In one embodiment, the inner shaft has a 0.018″ guidewire lumen. In some embodiments, a series of spiral cuts 1527 are formed along inner shaft 1525 in the proximal end 1526 proximal and distal to the inner shaft coupler 1530. Exemplary positioning of a series of spiral cuts 1527 are illustrated in the various embodiments of FIGS. 23A, 23B, 35, 36, and 37.

FIG. 16 is a distal end view of a bare scaffold 1510 showing three legs 1519 each terminating in a connection tab 1521.

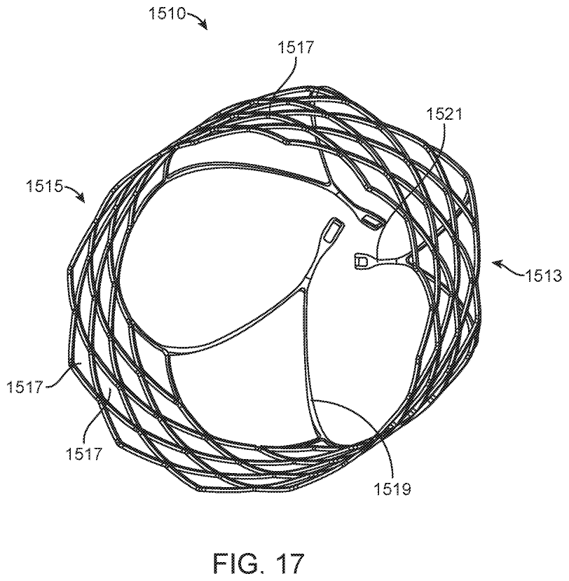

FIG. 17 is an isometric view of the bare scaffold 1510 of FIG. 16.

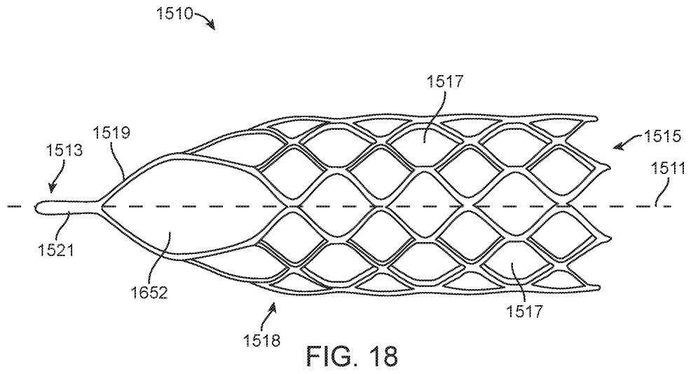

FIG. 18 is a side view of an exemplary scaffold structure having two legs 1519 only one visible in this view.

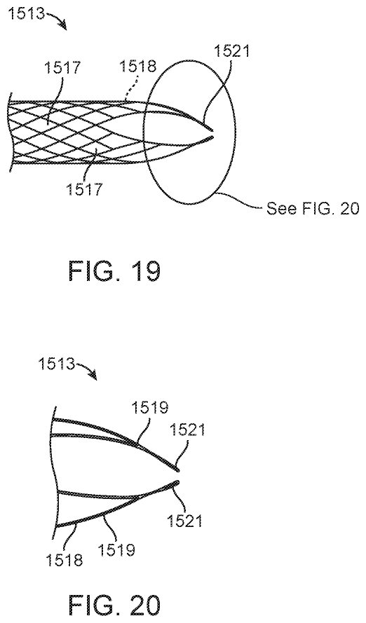

FIG. 19 is a side view of a bare scaffold 1510 with two legs 1519 for attachment to an inner shaft 1525 using an inner shaft coupler 1530.

FIG. 20 is an enlarged view of the connection tab 1521 on the end of each of the two legs 1519 of the scaffold embodiment of FIG. 19.

FIGS. 16, 17, 19, and 20 are distal, isometric, side and enlarged views, respectively, of a laser cut scaffold 1510 of a vascular occlusion device 1500. The covering, coating or membrane 1600 used to at least partially cover the scaffold is omitted to show the details of the scaffold. The scaffold 1510 may be formed from a cut tube of a biocompatible metal using a slot cut or a complex geometry cutting technique to provide a desired cell array as best seen in FIGS. 17, 19, 22, and 23A. The three legs 1519 structure shown in FIGS. 16, 17, 19 and 20 is provided as an exemplary benefit of the cutting pattern. The three legs could also be wires as in some embodiments the laser cut scaffold is not necessarily a one piece design. In some embodiments, the legs or other structure may be one or more separate pieces designed to address one or more performance features, like collapsing for optimal packing space, or a way to guide the membrane to a collapsed or constrained state.

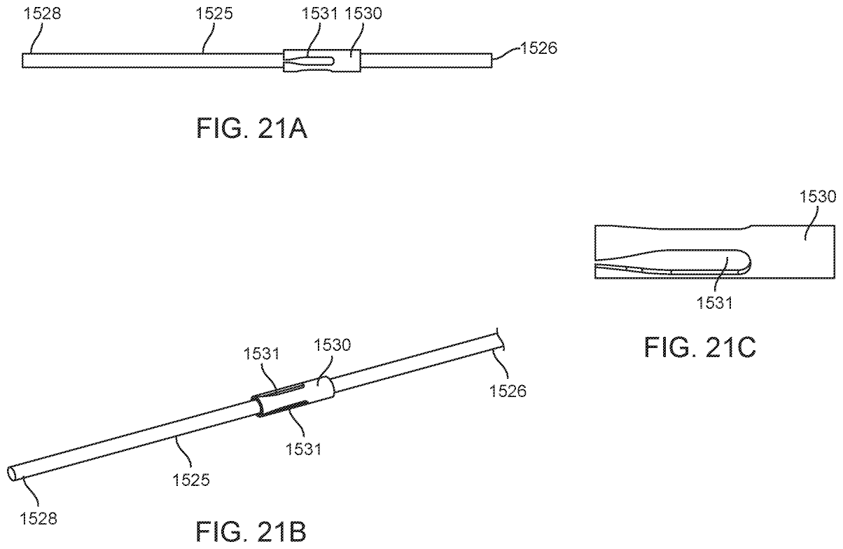

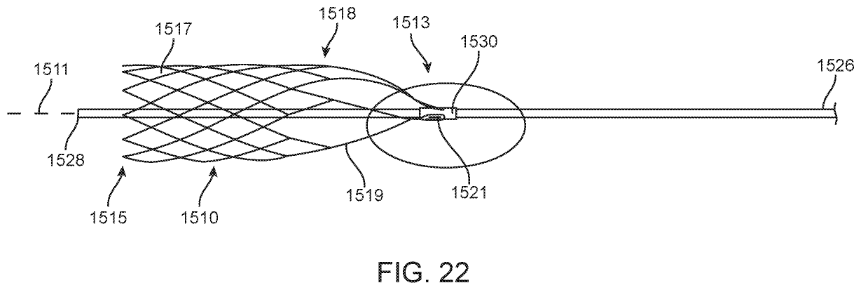

In one embodiment, the scaffold structure 1510 terminates in one end with leg connection tabs 1521 as shown in FIGS. 16, 17 and 20. In one aspect, the shape of the leg connection tabs 1521 is designed to be complementary with the corresponding slots or complementary key features 1531 formed in an inner shaft coupler 1530. FIGS. 21A, 21B and 21C illustrate isometric and side views respectfully of an exemplary inner shaft coupler 1530 to receive the leg connection tabs 1521. The connection tabs 1521 may be joined to the inner shaft coupler 1530 using any suitable joining technique such as welding or brazing. The final joint appears as shown in FIG. 22 or 23B with the legs 1519 of the scaffold device affixed to the inner shaft coupler 1530 which is affixed to the inner shaft 1525 or hypotube. Additionally or optionally, one or more notches, cuts or slots may be formed in the inner shaft 1525 in one or more locations to improve the flexibility of the inner shaft. In one embodiment, the inner shaft 1525 or hypotube is provided with a pattern of spiral cuts 1527 proximal to the inner shaft coupler 1530, distal to the inner shaft coupler 1530 or proximal and distal to the inner shaft coupler 1530 as needed to provide the desired flexibility in the inner shaft 1525. FIGS. 23A and 23B illustrate an embodiment of an exemplary spiral cut pattern 1527.

FIGS. 21A and 21B are side and perspective views, respectively, of the two key features 1531 of an inner shaft coupler that is attached to an inner shaft.

FIG. 21C is an enlarged view of the shaft coupler of FIGS. 21A and 21B showing the detail of a key feature 1531 shaped to engage with a connection tab 1521 of a scaffold leg 1519.

The inner shaft coupler 1530 is sized for placement on hypotube or central inner shaft 1525. The inner shaft coupler 1530 has keyed or complementary features 1531 to engage with the leg connection tabs 1521 of the scaffold. The proximal end features 1521 of the scaffold legs 1519 are keyed to mate with the inner shaft coupler 1530. The complementary cut outs 1531 used to join the leg tabs 1521 may come in a wide array of shapes and sizes to ensure orientation and position of the scaffold 1510 relative to the central or inner shaft 1525.

In the view of FIG. 22, the inner shaft 1525 and scaffold 1510 attached. In this embodiment, there are no spiral cuts 1527 on the inner shaft 1525. The scaffold covering 1600 is removed to show the scaffold detail. Also visible in this view is the joining the leg tabs 1521 and the inner shaft coupler 1530 to the hypotube or inner shaft 1525.

FIGS. 23A and 23B illustrate details of a series of spiral cuts 1527 made in the inner shaft 1525 proximal and distal to the inner shaft coupler 1530. Also visible in this view is the joining of the leg tabs 1521 and the inner shaft coupler 1530 to the hypotube or inner shaft 1525.

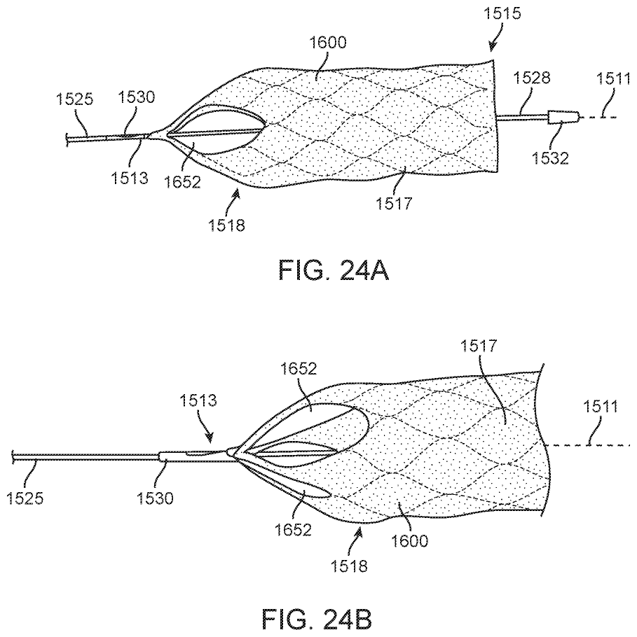

FIG. 24A is an exemplary view of a covered scaffold in a deployed configuration connected to the inner shaft. Openings 1652 cut around the legs and the atraumatic tip 1532 of the inner shaft are also visible in this view.

FIG. 24B is an enlarged view of the proximal end of the covered scaffold in FIG. 24A showing the covering 1600 on the legs 1519 extends into the inner shaft coupler 1530. This view also shows the cut outs 1652 formed in the covering 1600 between the covered legs of the scaffold.

FIGS. 24A and 24B include the one or more openings 1652 are formed in the covering. The openings 1652 in FIGS. 24A and 24B allow for the scaffold transition zone 1518 and the legs 1519 to remain covered while providing large openings to permit perfusion blood flow through the covered scaffold.

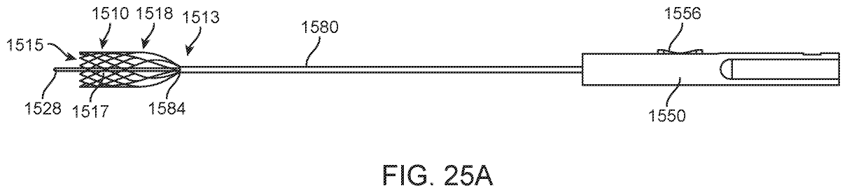

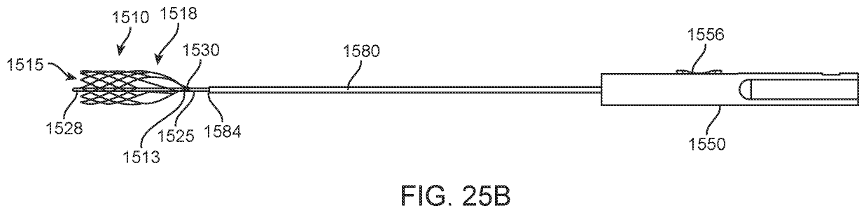

FIG. 25A is a side view of a vascular occlusion device shown without any cover. In this view, the outer shaft is withdrawn using the slider on the handle to position the distal end of the outer shaft at the proximal end of the scaffold. In this embodiment, in the deployed configuration the outer shaft is withdrawn proximal to the scaffold transition zone with the inner shaft coupler remaining within and covered by the outer shaft.

FIG. 25B is a side view of a vascular occlusion device of FIG. 25A. The slider on the handle is in a proximal position to withdraw the outer shaft or sheath from the scaffold allowing the scaffold to transition from a stowed configuration to deployed configuration as illustrated. In this embodiment, in the deployed configuration the outer shaft is withdrawn proximal to the inner shaft coupler.

FIG. 25A is a side view of an exemplary vascular occlusion device, with covering removed to show scaffold detail. There is a handle 1550 coupled to the inner and outer shafts 1525, 1580. An outer shaft or sheath 1580 is disposed over the inner shaft and the scaffold structure and is moveable by a slider on the handle. A slider in the handle controls the position of the outer shaft 1580 or sheath relative to the inner shaft 1525 and scaffold 1510. The slider knob 1556 is shown in a proximal position on the handle. In this position the sheath is moved proximally towards the handle thereby allowing the scaffold to transition from the stowed configuration to the deployed configuration. In the deployed configuration the vascular occlusion device engages the vessel interior wall to seal partially or completely as desired by the amount of occlusion and distal perfusion to be achieved by a specific embodiment. FIG. 25B is another view of the device in FIG. 25A with the guide partially withdrawn to show the detail of the spiral cuts on the hypotube proximal and distal to the mating collar.

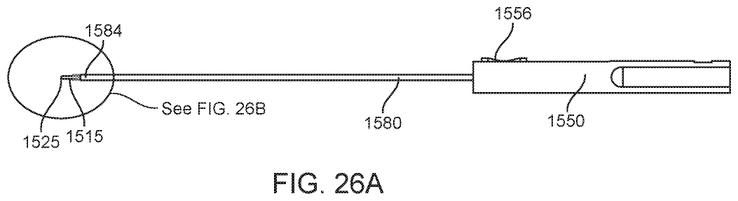

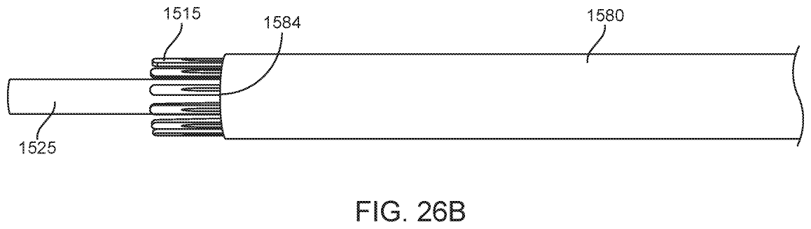

FIG. 26A is a side view of a vascular occlusion device in a stowed condition with the outer shaft withdrawn slightly to show the stowed distal end of the scaffold as best seen in the enlarged view of FIG. 26B. The slider on the handle is withdrawn slightly from the distal most position on the handle to only slightly withdraw the outer sheath to the illustrated position. Continued proximal movement of the slider will continue to withdraw the outer shaft or sheath from the scaffold allowing the scaffold to transition from a stowed configuration to deployed configuration.

FIG. 26A shows an exemplary vascular occlusion device in a stowed configuration. The slider knob is in a distal position on the handle and the sheath is covering substantially all of the scaffold device. Slider knob 1556 is used to control position of sheath or outer shaft 1580—shown in position to maintain sheath over the scaffold which retains the scaffold 1510 in a stowed configuration. FIG. 26B is an enlarged portion of the distal end of the device shown in FIG. 26A. In the view of FIG. 26B, the distal end of the sheath terminates with the distal most end of the scaffold and the terminal end of the hypotube exposed. Other sheath positions are possible where the scaffold is maintained in a stowed configuration an only the terminal end or portion of the hypotube is exposed. Optionally, the sheath may be selected such that none of the hypotube or the scaffold is showing. In additional embodiments, the sheath is positioned relative to the stowed condition of the scaffold to allow for ease of movement of the slider to deploy the scaffold.

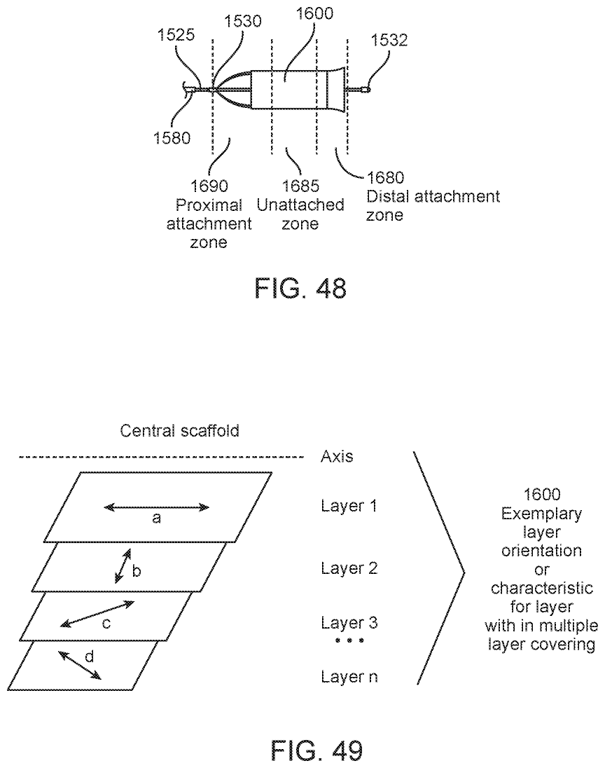

It is to be appreciated that a number of different scaffold coverings 1600 may be provided that will provide for at least partial occlusion of the peripheral vessels while simultaneously providing for perfusion blood flow to the vessels and structures distal to the vascular occlusion device. Additional details of the scaffold covering 1600 are described below with regard to FIGS. 48 and 49.

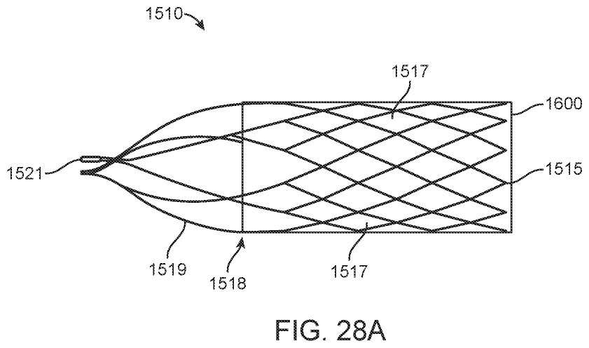

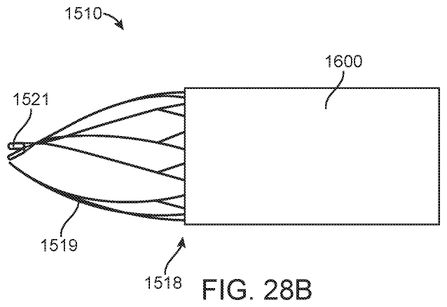

FIGS. 27, 28A and 28B are isometric and side views respectively of a scaffold device that covers most all of the scaffold structure from the distal end 1513 to the proximal end 1513 including the legs 1519 and connection tabs 1521 in some embodiments. When deployed within the vasculature the covered portion of the scaffold is one factor used to refine and define occlusion characteristics of the device. While once the covered scaffold is deployed in the vasculature, the blood flow is directed into the interior of the scaffold through the open central portion along the central longitudinal axis 1511 of the scaffold as well as through other uncovered or only partially covered scaffold portions are also used to refine and define the vascular occlusion device perfusion characteristics. Adjusting the relative amount and type of covering and open scaffold portions enables a wide array of occlusion and perfusion device characteristics. In some embodiments, a covered scaffold in the cylindrical scaffold portion extends from distal most portion of scaffold but covering stops before transition to legs in the scaffold transition zone 1518. An interior wall of the scaffold covering or membrane is also visible in the view of FIG. 27.

In some alternative embodiments, all of the scaffold structure but the legs are covered by a suitable scaffold covering 1600. The distal end to a portion of the scaffold where the legs are extending towards the coupling device as detailed above. In this way, some scaffold embodiments deploy into much like a tube or barrel shape which extends along the adjacent vessel wall where the scaffold is deployed. Any peripheral vessel along the covered portion of the main vessel will be partially or fully occluded. The covering extends from the distal end of the scaffold structure to the proximal end where the scaffold structure transitions to the legs and then tabs for joining to the coupling on the inner tube. The scaffold covering 1600 is shown as transparent in the view of FIG. 28A in show the detail of the scaffold structure in relation to the size of scaffold covering used. The scaffold covering 1600 material may be transparent or opaque. An opaque membrane or scaffold covering is shown in FIG. 28B.

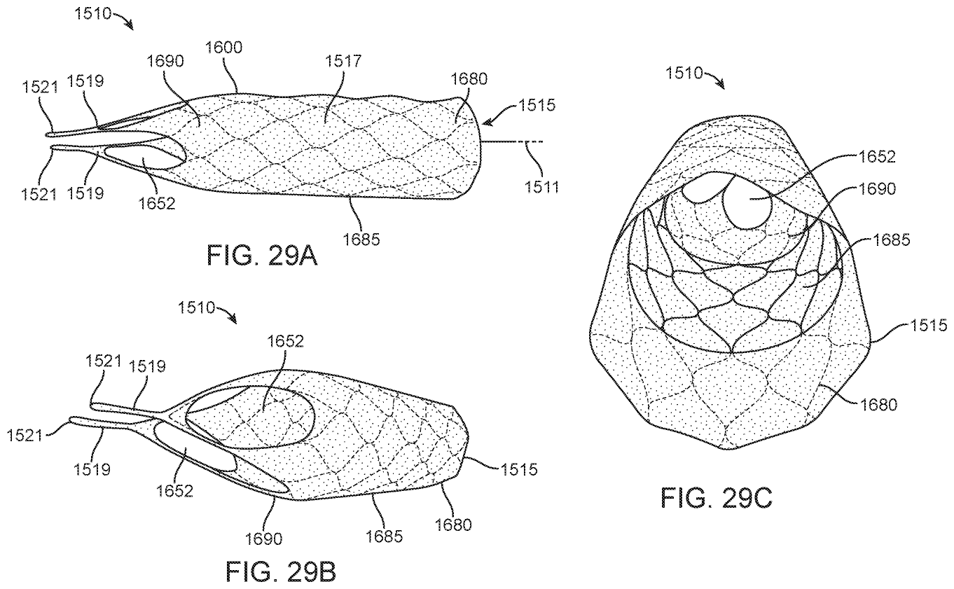

FIG. 29A is a side view of a covered scaffold embodiment having two legs for attachment to the central shaft. This covered scaffold embodiment includes a proximal scaffold attachment zone 1690, a distal scaffold attachment zone 1680 and a central covering portion that is unattached to the scaffold (unattached zone 1685). The covering 1600 on the legs to the connection tabs and the distal openings are also seen in this view.

FIG. 29B is a perspective view of the proximal end of the covered scaffold of FIG. 29A. The proximal attachment zone is visible in this view through a distal opening.

FIG. 29C is a perspective view of the distal end of the covered scaffold in FIG. 29A. The proximal attachment zone, the distal attachment zone and the distal openings are visible in this view. In one embodiment, the distal and proximal attachment portions are formed by folding the scaffold covering over the proximal and distal ends of the scaffold. FIG. 29 also illustrates a distal end 1620 that includes a distal folded portion 1622 over the distal end of the scaffold 1515. Similarly, a proximal end 1630 may include a proximal folded portion 1632 over the proximal end of the scaffold 1513, optionally including covering the legs 1519 and optionally including covering the connection tabs 1521.

FIGS. 29A, 29B and 29C include the one or more openings 1652 are formed in the scaffold covering 1600. The openings 1652 best seen in FIGS. 29A and 29B allow for the scaffold transition zone 1518 and both of the legs 1519 to remain covered while providing large openings to permit perfusion blood flow through the covered scaffold.

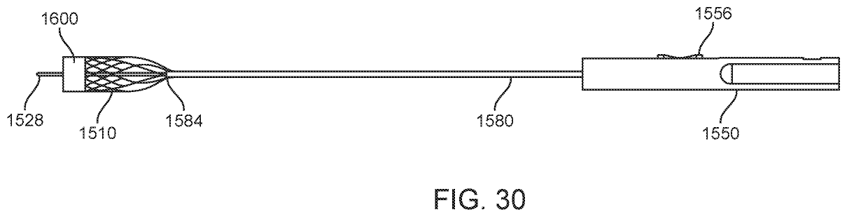

FIG. 30 is a side view of an exemplary vascular occlusion device, with a 20% covering of the scaffold. There is a handle coupled to a hypotube. A sheath is disposed over the hypotube and coupled to the handle. A slider knob in the handle controls the position of the sheath relative to the hypotube and scaffold device. The slider knob is shown in a proximal position on the handle. In this position the sheath is moved proximally towards the handle thereby allowing the scaffold to transition from the stowed configuration to the deployed configuration. In the deployed configuration the vascular occlusion device engages the vessel interior wall to seal partially or completely as desired by the amount of occlusion and distal perfusion to be achieved by a specific embodiment. Full Device 20% covered scaffold. Distal end of the covering aligns to the distal most portion of the scaffold structure. Slider to control position of sheath—shown in position to retract the sheath. Proximal end of the covering extends along the scaffold structure so that approximately 20% of the scaffold structure is covered. When deployed within the vasculature the covered portion of the scaffold is one factor used to refine and define occlusion characteristics of the device while the generally open central portion or other uncovered scaffold portions refine and define the device perfusion characteristics. Adjusting the relative amount and type of covering and open scaffold portions enables a wide array of occlusion and perfusion device characteristics (FIG. 30).

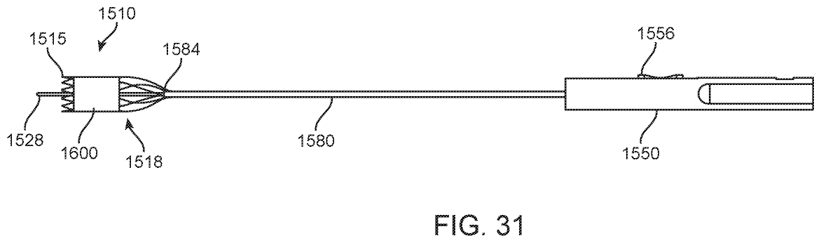

FIG. 31 is a side view of an embodiment of a vascular occlusion device in a deployed condition having a 50% scaffold covering. The slider on the handle is in a proximal position to withdraw the outer shaft or sheath from the scaffold allowing the scaffold to transition from a stowed configuration to deployed configuration as illustrated. The 50% scaffold covering distal end aligns proximal to the scaffold distal end and extends proximally along the longitudinal length of the scaffold to cover approximately 50% of the overall length of the scaffold.

FIG. 31 is a side view of an exemplary vascular occlusion device, with a 50% covering of the scaffold. There is a handle coupled to a hypotube. A sheath is disposed over the hypotube and coupled to the handle. A slider knob in the handle controls the position of the sheath relative to the hypotube and scaffold device. The slider knob is shown in a proximal position on the handle. In this position the sheath is moved proximally towards the handle thereby allowing the scaffold to transition from the stowed configuration to the deployed configuration. In the deployed configuration the vascular occlusion device engages the vessel interior wall to seal partially or completely as desired by the amount of occlusion and distal perfusion to be achieved by a specific embodiment. Full device—50% coverage centered. When deployed within the vasculature the covered portion of the scaffold is one factor used to refine and define occlusion characteristics of the device while the generally open central portion or other uncovered scaffold portions refine and define the device perfusion characteristics. Adjusting the relative amount and type of covering and open scaffold portions enables a wide array of occlusion and perfusion device characteristics. Distal end of the covering is spaced back proximally from the distal most end (the crowns) of the scaffold structure. Slider to control position of sheath—shown in position to retract the sheath. Proximal end of the covering extends along the scaffold structure so that approximately 50% of the scaffold structure is covered. The distal end of the covering is positioned along the scaffold structure and distal to the scaffold transition zone (FIG. 31).

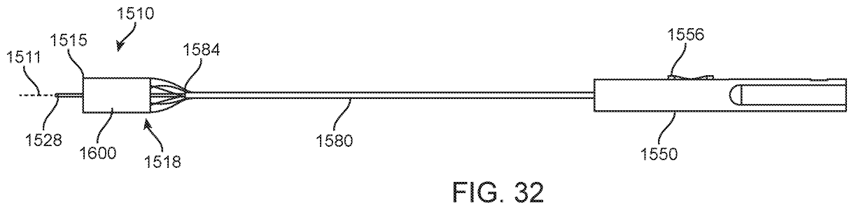

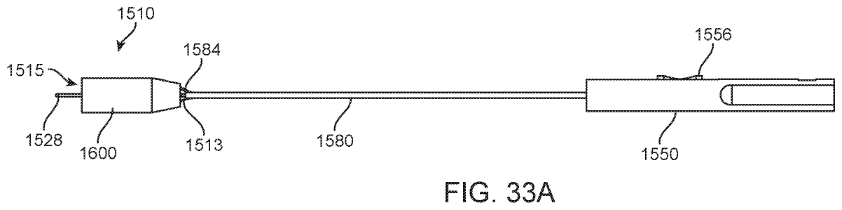

FIG. 32 is a side view of an embodiment of a vascular occlusion device in a deployed condition having an 80% scaffold covering. The slider on the handle is in a proximal position to withdraw the outer shaft or sheath from the scaffold allowing the scaffold to transition from a stowed configuration to deployed configuration as illustrated. The 80% scaffold covering distal end aligns with the scaffold distal end and extends proximally along the longitudinal length of the scaffold to cover approximately 80% of the overall length of the scaffold.

FIG. 32 is a side view of an exemplary vascular occlusion device, with an 80% covering of the scaffold. There is a handle coupled to a hypotube. A sheath is disposed over the hypotube and coupled to the handle. A slider knob in the handle controls the position of the sheath relative to the hypotube and scaffold device. The slider knob is shown in a proximal position on the handle. In this position the sheath is moved proximally towards the handle thereby allowing the scaffold to transition from the stowed configuration to the deployed configuration. In the deployed configuration the vascular occlusion device engages the vessel interior wall to seal partially or completely as desired by the amount of occlusion and distal perfusion to be achieved by a specific embodiment. Full device—80% coverage. Distal end of the covering aligns to the distal most portion of the scaffold structure. Slider to control position of sheath—shown in position to retract the sheath. Proximal end of the covering extends along the scaffold structure so that approximately 80% of the scaffold structure is covered. The distal end of the covering is positioned along the scaffold structure and terminates at the scaffold transition zone. The legs are uncovered. When deployed within the vasculature the covered portion of the scaffold is one factor used to refine and define occlusion characteristics of the device while the generally open central portion or other uncovered scaffold portions refine and define the device perfusion characteristics. Adjusting the relative amount and type of covering and open scaffold portions enables a wide array of occlusion and perfusion device characteristics (FIG. 32).

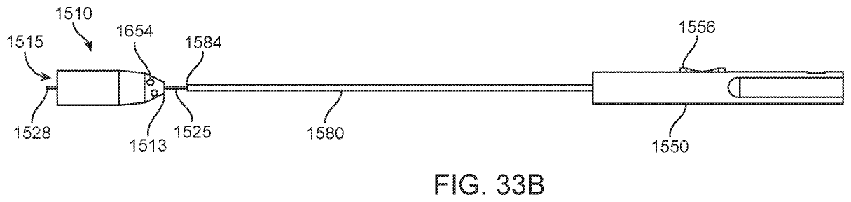

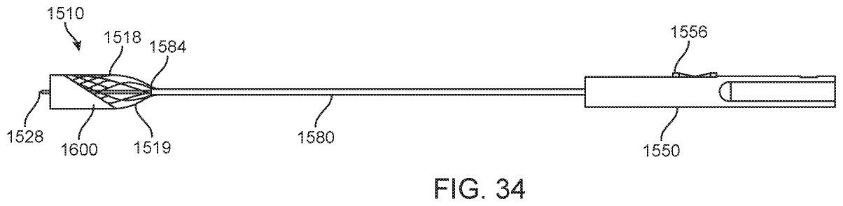

FIG. 33A is a side view of an embodiment of a vascular occlusion device in a deployed condition having an 100% scaffold covering. The 100% scaffold covering distal end aligns with the scaffold distal end and extends proximally along the longitudinal length of the scaffold to cover approximately 100% of the overall length of the scaffold with the exception of a small portion of the end of the device as shown. The slider on the handle is in a proximal position to withdraw the outer shaft or sheath from the scaffold allowing the scaffold to transition from a stowed configuration to deployed configuration as illustrated.

FIG. 33A is side view of a nearly completely covered vascular occlusion device. The embodiment of FIG. 33A is an exemplary vascular occlusion device, with a nearly 100% covering of the scaffold. The amount of distal perfusion may be adjusted by the gap between the covering around the proximal end of the device and the hypotube. There is a handle coupled to a hypotube. A sheath is disposed over the hypotube and coupled to the handle. A slider knob in the handle controls the position of the sheath relative to the hypotube and scaffold device. The slider knob is shown in a proximal position on the handle. In this position the sheath is moved proximally towards the handle thereby allowing the scaffold to transition from the stowed configuration to the deployed configuration. In the deployed configuration the vascular occlusion device engages the vessel interior wall to seal partially or completely as desired by the amount of occlusion and distal perfusion to be achieved by a specific embodiment. Full device—100% coverage scaffold with central flow through distal perfusion capability. Distal end of the covering aligns to the distal most portion of the scaffold structure. When deployed within the vasculature the covered portion of the scaffold is one factor used to refine and define occlusion characteristics of the device while the generally open central portion or other uncovered scaffold portions refine and define the device perfusion characteristics. Adjusting the relative amount and type of covering and open scaffold portions enables a wide array of occlusion and perfusion device characteristics. Proximal end of the covering extends along the scaffold structure so that approximately all of the scaffold structure is covered. The distal end of the covering is positioned along the scaffold structure and the transition portion. The legs are covered. The covering terminates along the legs leaving an opening of larger diameter than the sheath which allows a central distal perfusion flow. Small opening here—end is not closed. Slider to control position of sheath—shown in position to retract the sheath (FIG. 33A).

FIG. 33B is side view of a nearly completely covered vascular occlusion device. The embodiment of FIG. 33B is similar to that of FIG. 33A in that the vascular occlusion device has a nearly 100% covering of the scaffold. As with the embodiment of FIG. 33A, the amount of distal perfusion may be adjusted by the gap between the covering around the proximal end of the device and the hypotube. Additionally, the embodiment of FIG. 33B includes one or more apertures in the membrane or covering to further adjust the amount of distal perfusion.

FIG. 33B is a side view of an embodiment of a vascular occlusion device in a deployed condition having an 100% scaffold covering similar to FIG. 33A. The slider on the handle is in a proximal position to withdraw the outer shaft or sheath from the scaffold allowing the scaffold to transition from a stowed configuration to deployed configuration as illustrated. This embodiment illustrates a plurality of openings formed in the proximal end of the covering within the scaffold transition zone. The 100% scaffold covering distal end aligns with the scaffold distal end and extends proximally along the longitudinal length of the scaffold to cover approximately 100% of the overall length of the scaffold.

Similar to other embodiments, there is a handle on the proximal end of the vascular occlusion device. A sheath or outer shaft is disposed over the inner shaft or hypotube and coupled to the handle. A slider knob in the handle controls the position of the sheath relative to the hypotube and scaffold device. In this view, the slider knob is shown in a proximal position on the handle. In this position the sheath is moved proximally towards the handle thereby allowing the scaffold to transition from the stowed configuration to the deployed configuration. In the deployed configuration the vascular occlusion device engages the vessel interior wall to seal partially or completely as desired by the amount of occlusion and distal perfusion to be achieved by a specific embodiment.

In this embodiment, the full scaffold device is covered completely or considered a 100% coverage of the scaffold with the scaffold covering 1600. Advantageously, the directed flow through or distal perfusion capability is adjustable by the number, size and arrangement of the openings 1654 as shown in FIG. 33B. The amount of perfusion provided by the vascular occlusion device being determined by shape, size, pattern and location of perfusion openings or apertures 1654. While illustrated in the proximal end of the covered scaffold the apertures 1654 may be positioned in other portions of the scaffold covering 1600 depending on the clinical scenario where the vascular occlusion device is employed. As such, it is to be appreciated that a scaffold covering 1600 or other suitable biocompatible vascular membrane includes one or more or a pattern of apertures 1654 that are shaped, sized or positioned relative to the scaffold structure to modify the amount of distal perfusion. Additionally or optionally, the suitable membrane or scaffold covering 1600 may include apertures 1654 having one or more regular or irregular geometric shapes arranged in a continuous or discontinuous pattern which is selected to adapt the distal perfusion flow profile of an embodiment of the vascular occlusion device.

Distal end of the covering aligns to the distal most portion of the scaffold structure. When deployed within the vasculature the covered portion of the scaffold is one factor used to refine and define occlusion characteristics of the device while the generally open central portion or other uncovered scaffold portions refine and define the device perfusion characteristics. Adjusting the relative amount and type of covering and open scaffold portions enables a wide array of occlusion and perfusion device characteristics. Proximal end of the covering extends along the scaffold structure so that approximately all of the scaffold structure is covered. The distal end of the covering is positioned along the scaffold structure and the transition portion. The legs are covered. Distal perfusion is provided by flow through perfusion apertures formed in the membrane covering. Perfusion apertures may be provided as a pattern of small openings in the scaffold covering. Slider is used to control position of over shaft or sheath and is shown in position to retract the outer shaft.

FIG. 34 is a side view of an embodiment of a vascular occlusion device in a deployed condition having a tapered scaffold covering of a partial cylindrical section. The slider on the handle is in a proximal position to withdraw the outer shaft or sheath from the scaffold allowing the scaffold to transition from a stowed configuration to deployed configuration as illustrated. The tapered scaffold covering distal end aligns with the scaffold distal end and extends proximally along the longitudinal length of the scaffold to various distal positions according to the overall covering shape. In this view the exemplary shaped covering extends over only a few cells of the scaffold in the top portion while covering most all of the cells and nearly reaching the scaffold transition zone in the bottom portion.

FIG. 34 is side view of a partially completely covered vascular occlusion device. The embodiment of FIG. 34 illustrates how the shape of the membrane or covering may be modified in order to adjust the amount of distal perfusion. In the embodiment of FIG. 34 there is a tapered cylindrical membrane attached to the scaffold. Other partially covered membrane shapes may be used including combinations of regular and irregular shapes to adapt the membrane and scaffold structure to the specific anatomical environment or a desired occlusion and distal perfusion flow profile. As such, the amount of distal perfusion may be adjusted by the relative amounts of covered and exposed scaffold. Additionally or optionally, the shaped membrane embodiment of FIG. 34 may include one or more apertures in the membrane or covering to further adjust the amount of distal perfusion. There is a handle coupled to the inner and outer shafts as described herein. The slider knob is shown in a proximal position on the handle. In this position the sheath is moved proximally towards the handle thereby allowing the scaffold to transition from the stowed configuration to the deployed configuration. In the deployed configuration the vascular occlusion device engages the vessel interior wall to seal partially or completely as desired by the amount of occlusion and distal perfusion to be achieved by a specific embodiment.

Occlusion and perfusion device embodiment with a partial scaffold covering or membrane. In some embodiments, the scaffold covering 1600 or membrane may also cover only a portion of the scaffold in any of a variety of shapes such as the cut cylinder shape shown here. Other geometric shapes or irregular shapes may be employed for membrane overall shapes which will enable a wide array of different and controllable occlusion parameters along with a variety of simultaneous distal perfusion capabilities. When deployed within the vasculature the covered portion of the scaffold is one factor used to refine and define occlusion characteristics of the device while the generally open central portion or other uncovered scaffold portions refine and define the device perfusion characteristics. Adjusting the relative amount and type of covering and open scaffold portions enables a wide array of occlusion and perfusion device characteristics (see FIG. 34).

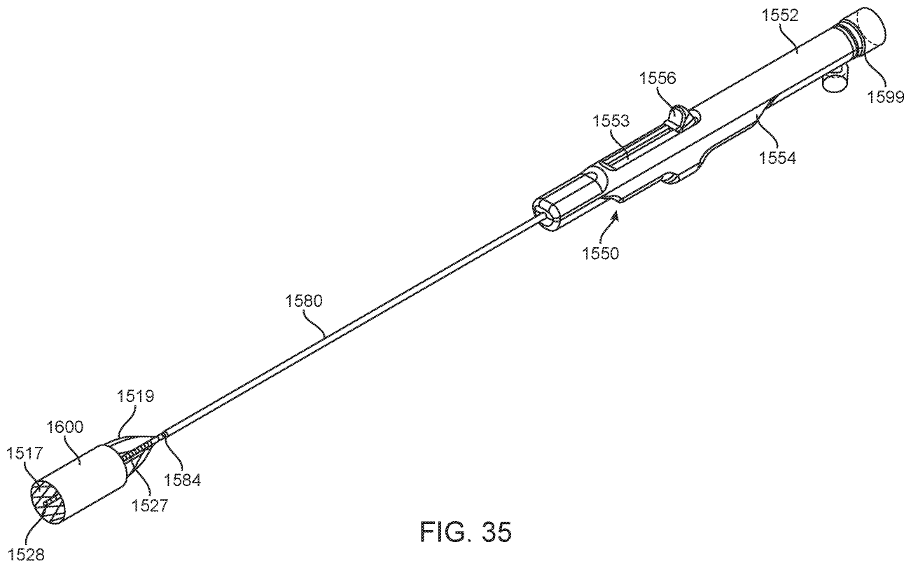

FIG. 35 is a perspective view of an embodiment of a vascular occlusion device in a deployed configuration having a scaffold covering extending from the distal end of the scaffold to the scaffold transition zone. The slider on the handle is in a proximal position to withdraw the outer shaft or sheath from the scaffold allowing the scaffold to transition from a stowed configuration to deployed configuration as illustrated. A portion of the distal attachment zone is visible in this view along with a section of the spiral cut inner shaft.

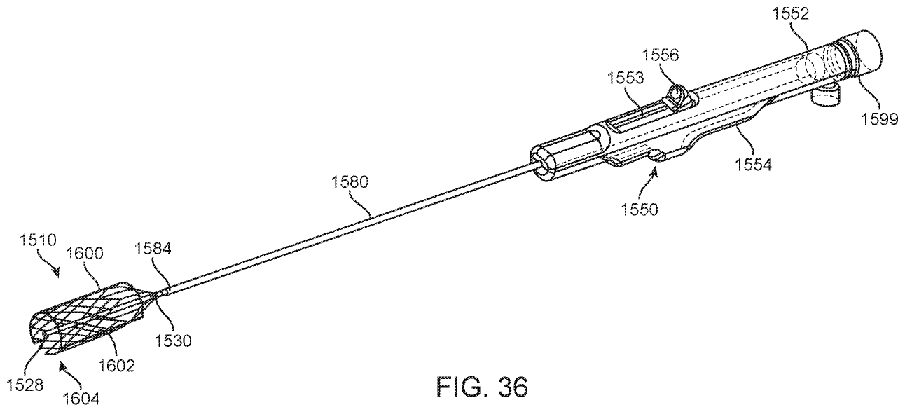

FIG. 36 is a perspective view of an embodiment of a vascular occlusion device in a deployed configuration having a scaffold covering extending from the distal end of the scaffold to the scaffold transition zone for about 270 degrees of the scaffold circumference. A portion of the scaffold along the bottom section remains uncovered as shown. The slider on the handle is in a proximal position to withdraw the outer shaft or sheath from the scaffold allowing the scaffold to transition from a stowed configuration to deployed configuration as illustrated. A portion of the distal attachment zone is visible in this view along with a section of the spiral cut inner shaft.

The vascular occlusion device of FIG. 36 is an exemplary embodiment of an occlusion device where the scaffold covering extends partially circumferentially about the scaffold structure. As seen in this view, the scaffold covering extends from the distal attachment zone 1680 to the proximal attachment zone 1690 and also includes an uncovered scaffold structure 1604. In this exemplary embodiment, the scaffold covering 1600 has a partial circumferential portion 1602 that extends partially circumferentially about 270 degrees of the scaffold structure from the distal attachment zone to the proximal attachment zone with the uncovered portion 1604 along the bottom of the scaffold. An embodiment such as this would be useful for peripheral vessels that are located on the sidewalls or upper portion of the vessel.

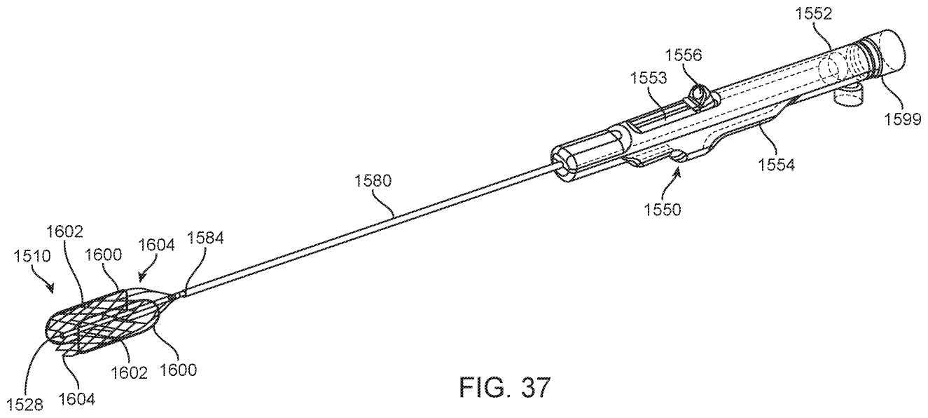

FIG. 37 is a perspective view of an embodiment of a vascular occlusion device in a deployed configuration having a pair of scaffold covering sections 1602 extending from the distal end of the scaffold to the scaffold transition zone for about 45 degrees of the scaffold circumference. Upper and lower uncovered scaffold portions 1604 are along the top and bottom of the scaffold. The portions 1604 of the scaffold along the top and bottom section remains uncovered as shown. The slider 1556 on the handle 1550 is in a proximal position to withdraw the outer shaft 1580 or sheath from the scaffold allowing the scaffold to transition from a stowed configuration to deployed configuration as illustrated. An embodiment such as this would be useful for peripheral vessels that are located on the sidewalls of the vessel.

A portion of the distal and proximal attachment zones of one of the scaffold covering sections is visible in this view along with a section of the spiral cut inner shaft.

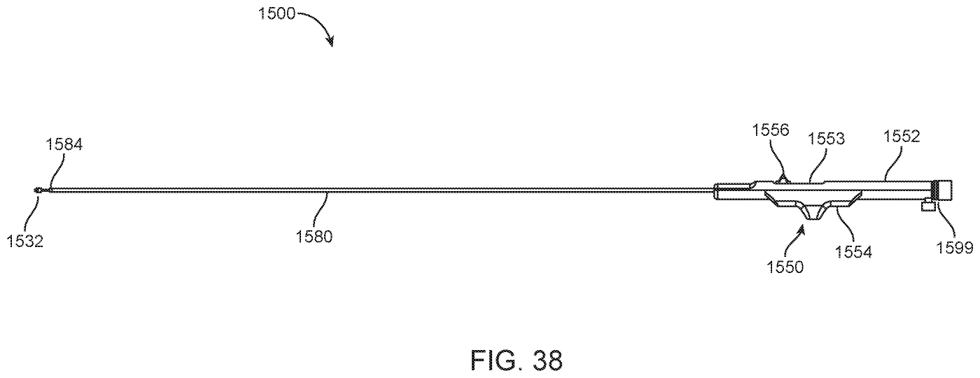

FIG. 38 is a perspective view of an embodiment of a vascular occlusion device in a stowed configuration. The slider on the handle is in a distal position with the outer shaft or sheath over the covered scaffold and maintaining it in a stowed configuration.

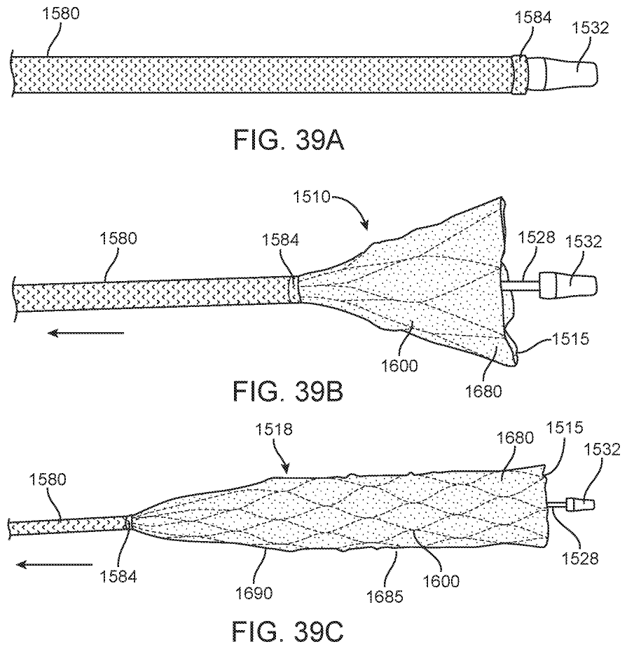

FIG. 39A is an enlarged view of the distal end of the stowed vascular occlusion device of FIG. 38.

FIG. 39B is the enlarged view of FIG. 39A showing the proximal movement (indicated by the arrows) of the distal end of the outer shaft 1580 or sheath as the slider on the handle advances proximally. The distal end of the covered scaffold and a portion of the distal attachment zone 1680 is also shown in this view.

FIG. 39C is the view of FIG. 39B showing the result of continued proximal movement of the slider (indicated by the arrows for the movement of the outer shaft 1580) and corresponding proximal movement of the outer shaft allowing more of the covered scaffold to transition into the deployed configuration.

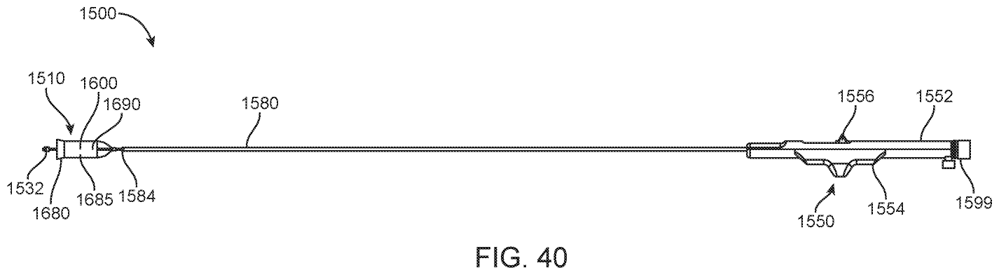

FIG. 40 is a perspective view of the vascular occlusion device of FIG. 38 after the slider is moved into the proximal position to fully transition the covered scaffold into the deployed configuration. The slider on the handle is in a proximal position with the outer shaft or sheath withdrawn from the covered scaffold which is shown in a deployed configuration.

FIG. 41 is a perspective view of the vascular occlusion device of FIG. 40 with a section of the outer shaft removed to position the deployed covered scaffold adjacent the handle with the slider shown in the proximal position to fully transition the covered scaffold into the deployed configuration as shown.

FIG. 41 also shows a side view of the handle 1550 with the slider knob or slider 1556 in a proximal position to withdraw the outer shaft and allow the scaffold structure to be in a deployed configuration as shown in FIG. 41. The handle 1550 includes an upper handle housing 1552 and a lower handle housing 1554. The hemostasis valve 1599 is also visible in this view.

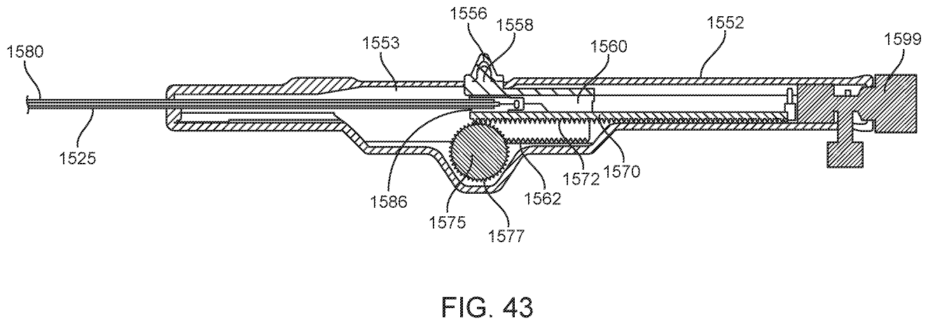

FIG. 42 is an exploded view of the handle embodiment of FIG. 41. A slider 1556 goes over tab 1558 on slider rack 1560. There is a slot 1553 in upper handle housing 1552 allows proximal and distal translation of slider 1556 (see FIG. 43). The slider rack 1560 has a tab 1558 used to engage with slider 1556 through slot 1553. The slider rack teeth 1562 are arranged to engage with inner gear 1579 on double gear pinion 1575. The outer shaft rack 1570 includes outer shaft rack teeth 1572. There is a receiver 1585 for engaging with outer shaft coupler 1586 on outer shaft 1580. Double gear pinion 1575 includes outer diameter teeth 1577 to engage with outer shaft rack teeth 1572 of outer shaft rack 1570. The double gear pinion includes inner diameter teeth 1579 to engage with the slider rack teeth 1562 of slider rack 1560. The outer shaft 1580 has a proximal end 1582 and a distal end 1584. The outer shaft coupler 1586 is adjacent to the outer shaft proximal end 1582 within the handle 1550. The double gear pinion and other components of the handle may be configured to provide a 3:1 gear ratio for transmitting the movement of the slide 1556 into translation of the outer sheath 1580.

FIG. 43 is a cross section view of the handle embodiment of FIG. 41. The tab 1558 is shown within the slider 1556 which is positioned in the proximal position within slot 1553. The spaced apart position of the receiver 1585 and the outer shaft coupler 1586 relative to the distal end of handle 1550 is also shown in this view. The outer shaft rack teeth 1572 are shown engaged with the outer diameter teeth 1579 of double gear pinion 1575.

In various embodiments, the occlusion system describe herein is compatible with other cardiac catheterization lab or interventional radiology lab workflow, designed with user-friendly functions and inserted and removed from patient similar to insertion of off-the-shelf introducer sheath with add-on function of temporary peripheral vascular occlusion. The device is an “assist device” which does not interfere with the standard catheterization procedure and comply with the standard activities in the catheterization lab.

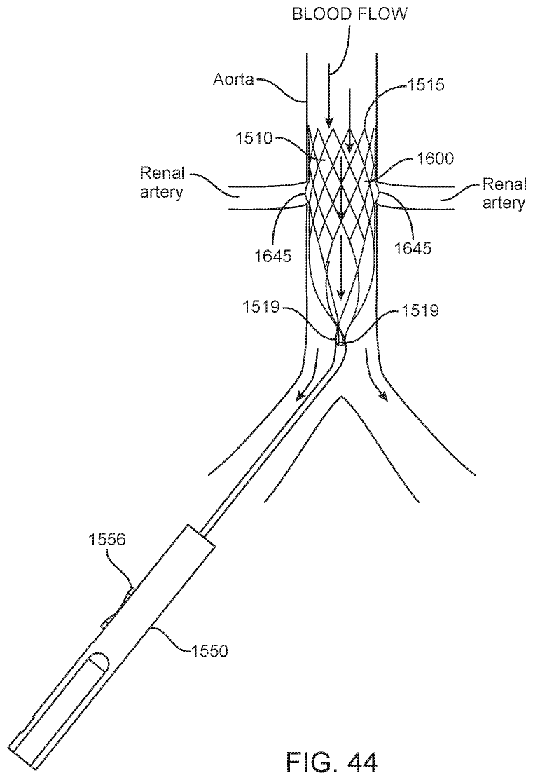

FIG. 44 is a cross section of a vascular occlusion device positioned for occlusion of the renal arteries and perfusion of the arterial tree in the lower extremities. This figure illustrates the distention or bulging 1645 of an unattached portion 1685 of the scaffold covering 1600 in response to the blood flow pressure generated within the scaffold 1510. As seen in this view, the unattached section 1685 of the scaffold covering is partially distended 1645 into and further ensuring the desired occlusion of the peripheral artery. In this illustrative embodiment, the temporarily occluded vessels are the renal arteries. Here, a portion of scaffold covering has bulged 1645 into and further occludes the renal artery ostia (see for example step 4640 in method 4600 or step 4740 in method 4700). While illustrated for use with the renal ostia, the position of the unattached zone 1685 relative to the scaffold 1510 as well as the amount or size of the unattached portion 1685 may be adapted based on the use of the vascular occlusion device 1500 when used with any of a wide array of peripheral structures while also allowing for perfusion flow beyond the temporarily occluded portion of the vasculature. Other exemplary peripheral vasculatures which may be additionally at least partially occluded using the bulging response 1645 of the unattached scaffold covering zone 1685 include, for example, a hepatic artery, a gastric artery, a celiac trunk, a splenic artery, an adrenal artery, a renal artery, a superior mesenteric artery, an ileocolic artery, a gonadal artery and an inferior mesenteric artery while simultaneously allowing perfusion flow through or around the at least partially covered scaffold structure to distal vessels and structures.

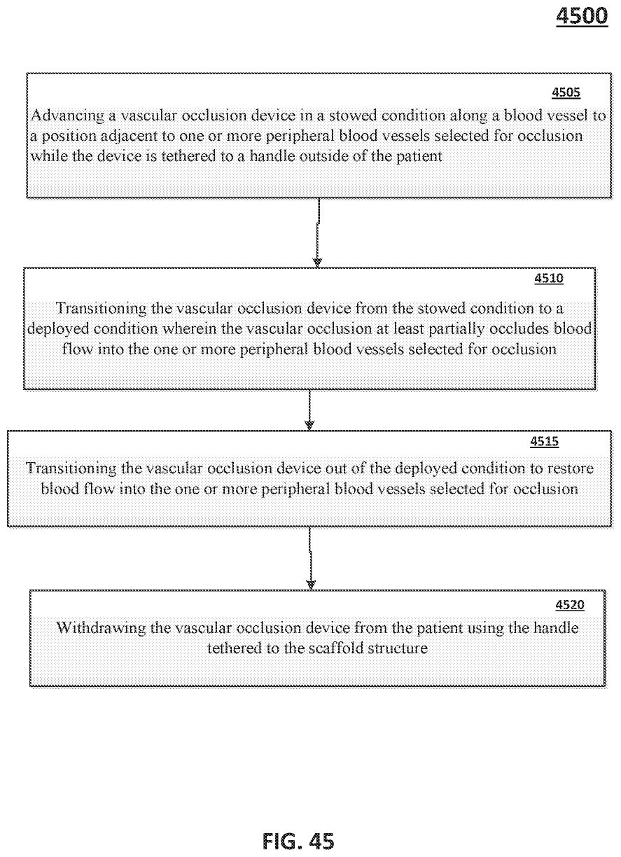

FIG. 45 is a flow chart of an exemplary method of providing occlusion with perfusion using an embodiment of a vascular occlusion device according to the method 4500.

First, at step 4505, there is the step of advancing a vascular occlusion device in a stowed condition along a blood vessel to a position adjacent to one or more peripheral blood vessels selected for occlusion while the device is tethered to a handle outside of the patient.

Next, at step 4510, there is the step of transitioning the vascular occlusion device from the stowed condition to a deployed condition wherein the vascular occlusion at least partially occludes blood flow into the one or more peripheral blood vessels selected for occlusion.

Next, at step 4515, there is the step of transitioning the vascular occlusion device out of the deployed condition to restore blood flow into the one or more peripheral blood vessels selected for occlusion.

Finally, at step 4520, there is the step of withdrawing the vascular occlusion device from the patient using the handle tethered to the scaffold structure.

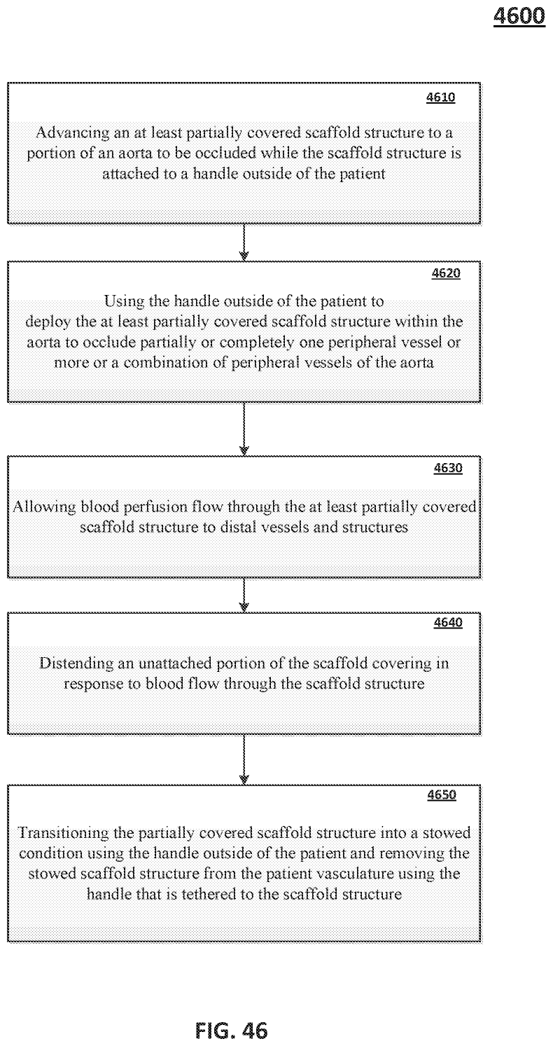

FIG. 46 is a flow chart of an exemplary method of providing occlusion with perfusion using an embodiment of a vascular occlusion device according to the method 4600.

First, at step 4610, there is the step of advancing an at least partially covered scaffold structure to a portion of an aorta to be occluded while the scaffold structure is attached to a handle outside of the patient.

Next, at step 4620, there is the step of using the handle outside of the patient to deploy the at least partially covered scaffold structure within the aorta to occlude partially or completely one peripheral vessel or more or a combination of peripheral vessels of the aorta.

Next, at step 4630, there is the step of allowing blood perfusion flow through the at least partially covered scaffold structure to distal vessels and structures.

Next, at step 4640, there is a step of distending an unattached portion of the scaffold covering in response to blood flow through the scaffold structure.

Next, at step 4650, there is a step of transitioning the partially covered scaffold structure into a stowed condition using the handle outside of the patient. Thereafter, removing the stowed scaffold structure from the patient vasculature using the handle that is tethered to the scaffold structure.

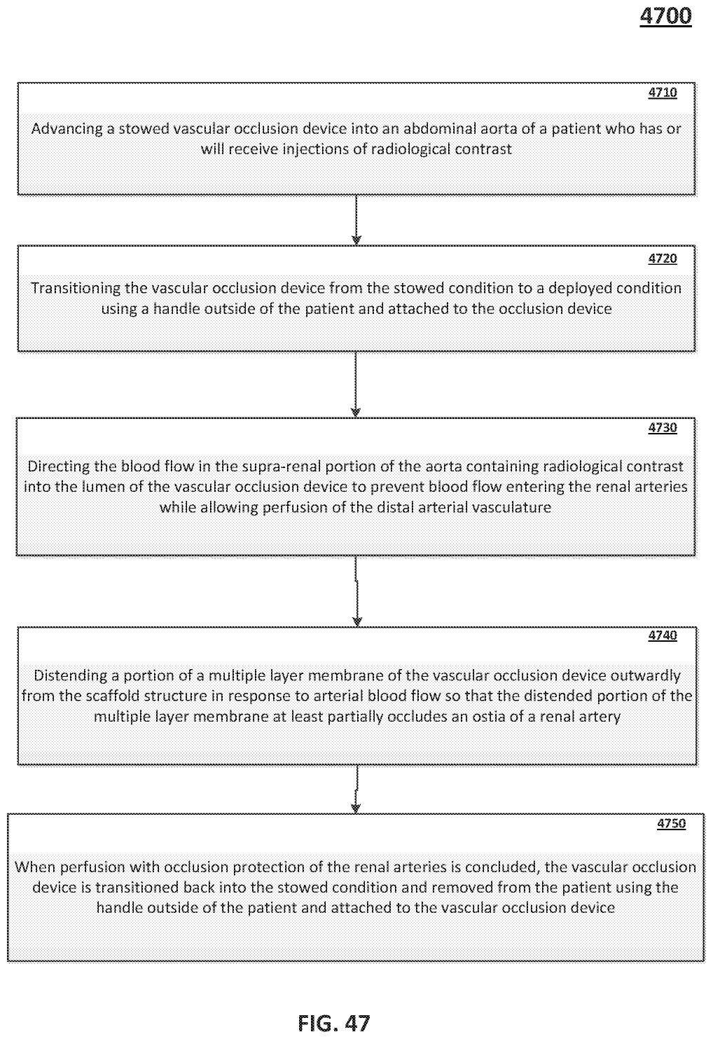

FIG. 47 is a flow chart of an exemplary method of providing occlusion with perfusion using an embodiment of a vascular occlusion device according to the method 4700.

First, at step 4710 there is a step of advancing a stowed vascular occlusion device into an abdominal aorta of a patient who has or will receive injections of radiological contrast.

Next, at step 4720, there is a step of transitioning the vascular occlusion device from the stowed condition to a deployed condition using a handle outside of the patient and attached to the occlusion device.

Next, at step 4730, there is a step of directing the blood flow in the supra-renal portion of the aorta containing radiological contrast into the lumen of the vascular occlusion device to prevent blood flow entering the renal arteries while allowing perfusion of the distal arterial vasculature.

Next, at step 4740, there is a step of distending a portion of a multiple layer membrane of the vascular occlusion device outwardly from the scaffold structure in response to arterial blood flow so that the distended portion of the multiple layer membrane at least partially occludes an ostia of a renal artery.

Next, at step 4750, there is a step performed when perfusion with occlusion protection of the renal arteries is concluded. At this point, the vascular occlusion device is transitioned back into the stowed condition and removed from the patient using the handle outside of the patient and attached to the vascular occlusion device.

FIG. 48 is a side view of an exemplary covered scaffold according to one embodiment of the vascular occlusion device. The covered scaffold indicates the distal attachment zone 1680, the proximal attachment zone 1690 and the unattached zone 1685 that indicate whether a portion of the scaffold covering 1600 is joined to the scaffold structure 1510 in that zone. The advantageous placement of the unattached zone 1685 allows embodiments of the covered scaffold to have a portion of the scaffold covering 1600 bulge or distend in response to blood flow. The distended scaffold covering 1600 may further occlude an adjacent peripheral vessel opening providing additional and targeted occlusion capabilities.

In some embodiments, the scaffold covering 1600 comprises a multiple layer structure that is attached to all or to select portions of the scaffold frame 1510. In some embodiments, the multiple layer covering is used to encapsulate all or a portion of the scaffold structure including the legs. The multiple layer scaffold covering may be a partial scaffold covering as seen in the embodiments of FIGS. 27, 28B, 30, 31, 32, 34, 35, 36 and 37 in relation to the percentage of the scaffold that is covered along the central axis 1511 or tapered in relation to the longitudinal axis as in FIG. 34. In one embodiment the scaffold distal end 1620 may include a distal folded portion 1622 over the distal end of the scaffold 1515. Along the same lines, the scaffold proximal end 1630 may include a proximal folded portion 1632 over the proximal end of the scaffold 1513, optionally including covering the legs 1519 and optionally including covering the connection tabs 1521. (See FIGS. 29A, 29B and 29C).

FIG. 49 is a partial exploded view of a portion of each of the individual layers that together form a multiple layer scaffold covering embodiment. Each one of the layers is shown with an arrow indicating an orientation of a characteristic or quality of that layer. Illustrated orientations are provided relative to the central axis of the scaffold structure as parallel (a), transverse (b) or oblique (c) or (d). In one embodiment, the orientation of each layer of the multi-layer structure determined by the predominant orientation of node and fibril microstructures within the layer as indicated by the arrows in FIG. 49. Additional details of adaptation of this characteristic of the multiple layer scaffold covering may be appreciated by reference to U.S. Pat. No. 8,840,824, incorporated herein by reference for all purposes. In still further embodiments, these or other characteristics of each of the layers of the multiple layer scaffold covering may be selected and positioned in the stack to further adapt characteristics such as strength, flexibility or permeability, as desired for a specific performance in an application of the vascular occlusion device.

In still other embodiments, any of the above described disturbing means such as a tunnel membrane illustrated and described in FIGS. 12A-13D may be covered using an embodiment of the scaffold covering 1600 including a multiple layer embodiment as well as the inclusion of the proximal and distal attachment zones and an unattached zone as described above. In still other embodiments, the embodiments shown of the occlusion with perfusion devices shown in FIGS. 19A-22B of US Patent Application Publication US 2018/0250015 may be modified to also include the scaffold covering and attachment and unattached zones described herein. It is to be appreciated that one or more of the layers of the multiple layers used to form the multiple layer embodiments of the scaffold layer 1600 may be selected from any of a wide array of suitable biocompatible materials including biocompatible soft or semi-soft plastics. The tunnel membrane previously described or the scaffold covering 1600 may comprise multiple individual layers of the covering material where one or more of the layers may differ from other layers. Additionally or optionally, the orientation of one or more layers used to form the scaffold covering may be selected so that in the aggregate multiple layer scaffold covering a desired characteristic or property of the scaffold covering, the covered scaffold, or the vascular device may better form the desired degree of occlusion with perfusion. In some embodiments one or more of the layers of a multiple layer scaffold covering 1600 is selected from one or more flexible films, ribbons, membranes such as polytetrafluoroethene (PTFE), fluorinated ethylene propylene (FEP), copolymers of hexafluoropropylene and tetrafluoroethylene, perfluoroalkoxy polymer resin (PFA), expanded polytetrafluoroethylene, silicone rubber, polyurethane, PET (polyethylene terephthalate), polyethylene, polyether ether ketone (PEEK), polyether block amide (PEBA), or other materials suited to the performance characteristics of the scaffold covering. In still other advantageous combinations of multiple layers of a scaffold covering, the layers used in the scaffold covering are selected to enhance the billowing or bulging response of an unattached zone in response to pressure waves within the blood flow. The billowing or bulging response may be modified based on the occlusion characteristics needed for selected peripheral vasculature where embodiments of the vascular occlusion devices with distal perfusion may be employed.

In view of the above, in other additional optional embodiments and configurations of the vascular occlusion devices described herein, an embodiment of a vascular occlusion device may be used to provide a method of providing occlusion of a portion of the vasculature of a patient with perfusion distal to the occlusion portion using the following method. First, there is a step of advancing a vascular occlusion device in a stowed condition along a blood vessel to a position adjacent to one or more peripheral blood vessels in the portion of the vasculature of the patient selected for occlusion while the vascular occlusion device is tethered to a handle outside of the patient. Next, there is a step of transitioning the vascular occlusion device from the stowed condition to a deployed condition using the handle wherein the vascular occlusion device at least partially occludes blood flow into the one or more peripheral blood vessels selected for occlusion. Next, the position of the vascular occlusion device which engages with the superior aspect of the vasculature to ensure that blood flow is directed into and along the lumen defined by the covered scaffold structure. As a result, the scaffold structure occludes the vessels targeted for temporary occlusion while directing the blood flow along the lumen of the vascular occlusion device through the interior of the covered scaffold to thereby maintain blood flow to blood vessels distal to the occluded portion of the vasculature. Furthermore, in some embodiments, the unattached zone of the covered scaffold deflects, bulges, or deforms in response to the blood flow now directed through the lumen of the covered scaffold. As a result, a portion of the unattached zone of the covered scaffold is urged into an adjacent opening of the peripheral blood vessel that is the target of the selected temporary occlusion procedure. It is to be appreciated that the location, size and number of unattached zones of a covered scaffold embodiment may vary according to the size, number and location of peripheral vessels selected for temporary occlusion. Thereafter, when the period of providing temporary occlusion is completed, the step of transitioning the vascular occlusion device from the deployed condition to the stowed condition using the slider on the handle which remains connected to the scaffold structure at all times during use. Once in the stowed configuration, the step of withdrawing the vascular occlusion device from the patient is performed by appropriate movement of the handle.

In another aspect, a method for mitigating exposure of the kidneys to medical contrast media is disclosed. The method comprises: inserting a catheter having a partially covered scaffold device into the vasculature and advancing into a desired position within an abdominal aorta; and deploying the scaffold so that the covering, membrane or tunnel structure is in a position to partially or complete occlude the renal arteries during use of contrast media while simultaneously providing perfusion blood flood distal to the occluding device. In certain embodiments, the insertion of the partially covered scaffold occlusion device to an aorta is accomplished by a transfemoral artery approach or by a trans-branchial artery approach or by a trans-radial artery approach. In some embodiments, the catheter and scaffold occlusion device are inserted along a guidewire and moved into a position to partially or completely occlude one or more blood vessels under appropriate medical imaging guidance such as fluoroscopy. Additional details and illustrations of the various vascular access routes described herein may be appreciated with reference to US Patent Application Publication US 2013/0281850 entitled, “Method For Diagnosis and Treatment of Artery,” which is incorporated herein by reference for all purposes. The above details and alternative method steps may also be applied to provide additional embodiments and variations to the steps detailed for methods 4500, 4600 and 4700 described herein.

Those of ordinary skill will appreciate that the devices and methods described herein meet the objective of a catheter based vascular occlusion system that will be able to be used to access the aorta with the ability to provide temporary occlusion of target vasculature while maintaining perfusion to the lower limbs vasculature. US Patent Application Publication US 2016/0375230 and US 2018/0250015 are incorporated herein by reference for all purposes.

The various embodiments of the vascular occlusion with perfusion devices described herein provide in a general way a flow disturbing means within the blood flow of the aorta. The distal most end of the scaffold engages substantially circumferentially with the interior wall of the aorta so that substantially all of the blood flow in the aorta flows into and along the central axis of the scaffold and out of the scaffold proximal openings. In one illustrative embodiment, a vascular occlusion device is positioned such that the scaffold or tunnel membrane shunts blood flowing from the supra-renal aorta through the scaffold or tunnel membrane, bypassing the renal arteries, and into the intra-renal aorta as the flow exits the scaffold. Alternative distal most segments of the scaffold may be used for greater contact area with the blood vessel where the vascular occlusion with perfusion device is employed. Optionally, the distal most segment of the scaffold may be in the shape of a flared distal end of the scaffold (see FIGS. 40 and 41). In an additional alternative embodiment, a flat distal engagement segment such as that exemplified by segment 1811 in FIG. 13A may also be used. Additionally or optionally, one or more flared segments, or one or more flat segments may be used alone or in combination to ensure fluid tight contact the wall of the supra-renal aorta and the wall of the infra-renal aorta, respectively if desired. Similar modifications may be made for use in other combinations of occlusion with perfusion on other possible peripheral vessels for clinical scenarios beyond protection of the kidneys from exposure to contrast agents. The aperture 106 may be substantially the same as the aperture 207 described previously herein. Regardless of the vascular occlusion embodiment selected, the shunting period or period of time that occlusion with perfusion is utilized may be (a) synchronized with the injection of a contrast media by a physician or (b) used so long as the occlusion of the selected peripheral vessel is clinically necessary however irrespective of length of use the scaffold remains attached to the handle which is outside of the patient's vasculature. In other words, the vascular occlusion devices that provide selective occlusion with perfusion are temporary vascular devices that are always tethered outside of the body during use. Still further, it is to be appreciated that the occlusion or shunting period should be kept to a minimum amount of time to shunt the contrast media but not long enough to cause renal ischemia by preventing blood flow to the kidneys. The kidneys are resistant to transient ischemia, therefore the shunting period may be tuned to avoid ischemia, depending upon the specific clinical situation where the device is being employed.

Exemplary Vascular Occlusion Devices and Covered Scaffolds

In some specific embodiments, the scaffold 1510 is fabricated as a laser cut tube of overall length from the connection tab 1521 on the legs 1519 to the scaffold distal end 1515 ranges from 40 mm to about 100 mm. Typically, the vascular occlusion device is delivered and maintained within a stowed configuration compressed with an 8 Fr compatible outer shaft or sheath. As best seen in FIG. 39A, the outer diameter of the outer sheath ranges from outer shaft overall diameter is between 0.100 inches and 0.104 inches. When the outer shaft is withdrawn as shown in FIG. 39C, the deployed condition of the covered scaffold structure into the vasculature, such as the lower aorta, has a deployed diameter ranging from 15 mm to 35 mm or an outer diameter ranging from 19 mm to 35 mm. As detailed in FIGS. 48 and 49, the scaffold covering may be formed from multiple layers of material to a final thickness of 0.001 inches in an unattached zone 1685 and 0.002 inches in each of the distal attached zone 1680 and the proximal attached zone 1690. Additionally, in other embodiments the vascular occlusion device may be characterized by the occlusive length of the deployed covered scaffold structure. The occlusive length of a covered scaffold structure is measured from the scaffold distal end 1515 to the distal end of the scaffold transition zone 1518 where the scaffold transitions to two or three or fewer legs and attachment to the inner shaft. In various embodiments the covered scaffold has an occlusive length ranging from 40 mm to 100 mm. In some embodiments, the vascular occlusion device has a 65 cm working length as measured from the handle 1550 to the distal end of the inner shaft 1528 and the atraumatic tip 1532.

Turning now to an exemplary bare scaffold structure as shown in FIG. 18. The scaffold cell geometry is laser cut into a tube and electropolished to a smooth finish. The resulting thickness of the scaffold is about 0.008″. There are typically 3 to 6 cells arranged along the longitudinal axis and 6 to 12 cells arranged along the perimeter. In general, a typical cell opening ranges from 1 cm to 2 cm along the longitudinal axis and from 0.5 cm to 1.5 cm along the circumference. In some embodiments, the cell orientation may be approximately diamond shaped when deployed with a major axis along the longitudinal axis of the scaffold and device in the range of 4 cm to 6 cm and a minor axis along the circumference of the device that ranges from 25 mm to 100 mm.

Although preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

1. A vascular occlusion device, comprising:
a. A handle having a slider;
b. An inner shaft coupled to the handle;
c. An outer shaft over the inner shaft and coupled to the slider;
d. A scaffold structure having a distal end, a scaffold transition zone and a proximal end having a plurality of legs wherein each leg of the plurality legs is coupled to a distal portion of the inner shaft, wherein the scaffold structure moves from a stowed configuration when the outer shaft is extended over the scaffold structure and a deployed configuration when the outer shaft is retracted from covering the scaffold structure; and
e. A scaffold covering over at least a portion of the scaffold structure, the multiple layer scaffold covering having a distal scaffold attachment zone where a portion of the scaffold covering is attached to a distal portion of the scaffold, a proximal scaffold attachment zone where a portion of the scaffold covering is attached to a proximal portion of the scaffold and an unattached zone between the distal attachment zone and the proximal attachment zone wherein the scaffold covering is unattached to an adjacent portion of the scaffold.

2. The vascular occlusion device of claim 1 wherein the plurality of legs is two legs or three legs.

3. The vascular occlusion device of claim 2 wherein the scaffold covering extends from the distal end of the scaffold structure to each of the two legs or the three legs.

4. The vascular occlusion device of claim 1 wherein the scaffold covering extends from the distal end of the scaffold structure proximally to cover approximately 20%, 50%, 80% or 100% of the overall length of the scaffold structure.

5. The vascular occlusion device of claim 1 wherein the scaffold covering extends completely circumferentially about the scaffold structure from the distal attachment zone to the proximal attachment zone.

6. The vascular occlusion device of claim 1 wherein the scaffold covering extends partially circumferentially about the scaffold structure from the distal attachment zone to the proximal attachment zone with an uncovered scaffold structure.

7. The vascular occlusion device of claim 6 wherein the scaffold covering extends partially circumferentially about 270 degrees of the scaffold structure from the distal attachment zone to the proximal attachment zone.

8. The vascular occlusion device of claim 6 wherein a first scaffold covering extends partially circumferentially about 45 degrees of the scaffold structure from the distal attachment zone to the proximal attachment zone and a second scaffold covering extends partially circumferentially about 45 degrees of the scaffold structure from the distal attachment zone to the proximal attachment zone, wherein the first scaffold covering and the second scaffold covering are on opposite sides of the longitudinal axis of the scaffold structure.

9. The vascular occlusion device of claim 1 wherein the multiple layer scaffold covering is attached to the scaffold in the distal scaffold attachment zone and in the proximal scaffold attachment zone by encapsulating a portion of the scaffold, by folding over a portion of the multiple layer scaffold covering and encapsulating a portion of the scaffold, by stitching the multiple layer scaffold covering to a portion of the scaffold, or by electrospinning the multiple layer scaffold to a portion of the scaffold.

10. The vascular occlusion device of claim 1 wherein the scaffold structure is formed from slots cut into a tube.

11. The vascular occlusion device of claim 1 wherein the covering is applied to nearly all, 80%, 70%, 60%, 50%, 30% or 20% of the scaffold structure.

12. The vascular occlusion device of claim 1 wherein scaffold covering is formed from multiple layers.

13. The vascular occlusion device of claim 12 wherein the layers of the multiple layer scaffold covering are selected from ePFTE, PTFE, FEP, polyurethane or silicone.

14. The vascular occlusion device of claim 1 wherein the scaffold covering or the more than one layers of a multiple layer scaffold covering is applied to a scaffold structure external surface, to a scaffold structure internal surface, to encapsulate the distal scaffold attachment zone and the proximal scaffold attachment zone, as a series of spray coats, dip coats or electron spin coatings to the scaffold structure.

15. The vascular occlusion device of claim 1 wherein the multiple layer scaffold covering has a thickness of 5-100 microns.

16. The vascular occlusion device of claim 1 wherein the multiple layer scaffold covering has a thickness of about 0.001 inches in an unattached zone and a thickness of about 0.002 inches in an attached zone.

17. The vascular occlusion device of claim 1 further comprising a double gear pinion within the handle that couples the outer shaft to the slider.

18. A method of providing selective occlusion with distal perfusion using a vascular occlusion device, comprising:
advancing the vascular occlusion device in a stowed condition along a blood vessel to a position adjacent to one or more peripheral blood vessels in the portion of the vasculature of the patient selected for occlusion while the vascular occlusion device is tethered to a handle outside of the patient;
transitioning the vascular occlusion device from the stowed condition to a deployed condition using the handle wherein the vascular occlusion device at least partially occludes blood flow into the one or more peripheral blood vessels selected for occlusion wherein the position of the vascular occlusion device engages with a superior aspect of the vasculature to direct blood flow into and along a lumen defined by a covered scaffold structure of the vascular occlusion device;
deflecting a portion of an unattached zone of the covered scaffold in response to the blood flow through the lumen of the covered scaffold into an adjacent opening of the one or more peripheral blood vessels in the portion of the vasculature of the patient selected for occlusion;
transitioning the vascular occlusion device from the deployed condition to the stowed condition using the handle; and
withdrawing the vascular occlusion device in the stowed condition from the patient.

19. The method of claim 18 wherein the one or more peripheral blood vessels in the portion of the vasculature of the patient selected for occlusion is selected from the group consisting of
a hepatic artery, a gastric artery, a celiac trunk, a splenic artery, an adrenal artery, a renal artery, a superior mesenteric artery, an ileocolic artery, a gonadal artery and an inferior mesenteric artery.

20. The method of claim 18 the covered scaffold unattached zone further comprising a position of a portion of the unattached zone to deflect into a portion of at least one of a hepatic artery, a gastric artery, a celiac trunk, a splenic artery, an adrenal artery, a renal artery, a superior mesenteric artery, an ileocolic artery, a gonadal artery and an inferior mesenteric artery when the vascular occlusion device is positioned within a portion of the aorta.

21. A method of temporarily occluding a blood vessel, comprising:
a. Advancing a vascular occlusion device in a stowed condition along a blood vessel to a position adjacent to one or more peripheral blood vessels selected for temporary occlusion;
b. Transitioning the vascular occlusion device from the stowed condition to a deployed condition wherein the vascular occlusion at least partially occludes blood flow into the one or more peripheral blood vessels selected for temporary occlusion while directing the blood flow through and along a lumen of a covered scaffold of the vascular occlusion device; and
c. Transitioning the vascular occlusion device out of the deployed condition to restore blood flow into the one or more peripheral blood vessels selected for temporary occlusion when a period of temporary occlusion is elapsed.

22. The method of claim 21 wherein directing the blood flow through and along the lumen of the vascular occlusion device maintains blood flow to components and vessels distal to the vascular occlusion device while at least partially occluding the blood flow to the one or more peripheral blood vessels.

23. The method of claim 21 wherein the one or more peripheral blood vessels are the vasculature of a liver, a kidney, a stomach, a spleen, an intestine, a stomach, an esophagus, or a gonad.

24. The method of claim 21 wherein the blood vessel is an aorta and the peripheral blood vessels are one or more or a combination of: a hepatic artery, a gastric artery, a celiac trunk, a splenic artery, an adrenal artery, a renal artery, a superior mesenteric artery, an ileocolic artery, a gonadal artery and an inferior mesenteric artery.

25. A method of reversibly and temporarily occluding a blood vessel, comprising:
a. Advancing an at least partially covered scaffold structure of a tethered vascular occlusion device to a portion of an aorta to be occluded; and
b. Using a handle of the vascular occlusion device to deploy the at least partially covered scaffold structure within the aorta to occlude partially or completely one or more or a combination of: a hepatic artery, a gastric artery, a celiac trunk, a splenic artery, an adrenal artery, a renal artery, a superior mesenteric artery, an ileocolic artery, a gonadal artery and an inferior mesenteric artery using a portion of a multiple layer scaffold covering while simultaneously allowing perfusion flow through a lumen of the at least partially covered scaffold structure to distal vessels and structures.

26. The method of claim 21 wherein the insertion of the vascular occlusion device or of the at least partially covered scaffold device to a blood vessel which is the aorta is introduced by transfemoral artery approach or by trans-brachial artery approach or by trans-radial artery approach.

27. The method of claim 21 further comprising: advancing the vascular occlusion device over a guidewire into a position adjacent to a landmark of the skeletal anatomy.

28. The method of claim 21 wherein a portion of an unattached zone of a multiple layer scaffold covering distends in response to blood flow along a lumen of the scaffold of the vascular occlusion device to occlude an opening of any of a hepatic artery, a gastric artery, a celiac trunk, a splenic artery, an adrenal artery, a renal artery, a superior mesenteric artery, an ileocolic artery, a gonadal artery and an inferior mesenteric artery.

29. A vascular occlusion device, comprising:
a. A handle having a slider knob;
b. An inner shaft coupled to the handle;
c. An outer shaft over the inner shaft and coupled within the handle to the slider knob;
d. A scaffold structure having at least two legs and a multiple layer scaffold covering, the at least two legs of the scaffold structure attached to an inner shaft coupler in a distal portion of the inner shaft;
e. The multiple layer scaffold covering positioned over at least a portion of the scaffold structure, wherein the scaffold structure moves from a stowed condition when the outer shaft is extended over the scaffold structure and a deployed condition when the outer shaft is retracted from covering the scaffold structure.

30. The vascular occlusion device of claim 29 wherein the scaffold structure is formed from slots cut into a tube.

31. The vascular occlusion device of claim 29 wherein the covering is applied to nearly all, 80%, 70%, 60%, 50%, 30% or 20% of the scaffold structure.

32. The vascular occlusion device of claim 29 wherein the multiple layer scaffold covering is made of ePFTE, PTFE, polyurethane, FEP or silicone.

33. The vascular occlusion device of claim 29 wherein the multiple layer scaffold covering is folded over a proximal portion and a distal portion of the scaffold.

34. The vascular occlusion device of claim 29 wherein after the multiple layer scaffold covering is attached to the scaffold, the scaffold further comprises a distal attachment zone, a proximal attachment zone and an unattached zone.

35. The vascular occlusion device of claim 29 wherein the multiple layer scaffold covering further comprises a proximal attachment zone, a distal attachment zone and an unattached zone wherein a thickness of the multiple layer covering in the proximal attachment zone and the distal attachment zone is greater than the thickness of the multiple layer scaffold covering in the unattached zone.

36. The vascular occlusion device of claim 35 wherein the multiple layer scaffold covering on the scaffold structure has a thickness of 5-100 microns.

37. The vascular occlusion device of claim 29 wherein scaffold structure has a cylindrical portion and a conical portion wherein the terminal ends of the conical portion are coupled to the inner shaft.

38. The vascular occlusion device of claim 29 wherein the inner shaft further comprises one or more spiral cut sections to increase flexibility of the inner shaft.

39. The vascular occlusion device of claim 38 wherein the one or more spiral cut sections are positioned proximally or distally or both proximal and distal to an inner shaft coupler where the scaffold structure is attached to the inner shaft.

40. The vascular occlusion device of claim 29 the scaffold structure further comprising two or more legs wherein each of the two or more legs terminates with a connection tab that is joined to a corresponding key feature on an inner shaft coupler.

41. The vascular occlusion device of claim 29 wherein the multiple layer scaffold covering includes one or more or a pattern of apertures that are shaped, sized or positioned relative to the scaffold structure to modify the amount of distal perfusion provided by the vascular occlusion device in use within the vasculature.

42. The vascular occlusion device of claim 29 wherein the multiple layer scaffold covering includes one or more regular or irregular geometric shapes arranged in a continuous or discontinuous pattern which is selected to adapt the distal perfusion flow profile of the vascular occlusion device in use within the vasculature.

43. The vascular occlusion device of claim 1 wherein when in a stowed configuration within the outer shaft the overall diameter is between 0.100 inches and 0.104 inches and when in a deployed configuration the covered scaffold has an outer diameter from 19 to 35 mm.

44. The vascular occlusion device of claim 1 wherein the covered scaffold has an occlusive length of 40 mm to 100 mm measured from a distal end of the scaffold to a scaffold transition zone.
